(19) United States
(12) Patent Application Publication
(10) Pub. No.: US 2006/0293395 A1
(43) Pub. Date: Dec. 28, 2006

(54) Methods for treating a mammal before, during and after cardiac arrest

(76) Inventors: Max Harry Weil, Rancho Mirage, CA (US); Shije Sun, Palm Desert, CA (US); Wauchan Tang, Palm Desert, CA (US); Robert J. Padley, Lake Bluff, IL (US); Leticia Delgado-Herrera, Lake Forest, IL (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 11/139,344

(22) Filed: May 27, 2005

Related U.S. Application Data
(60) Provisional application No. 60/575,765, filed on May 28, 2004.

(51) Int. Cl. A61K 31/122; A61N 1/00

(52) U.S. Cl. 514/682; 607/3

(57) ABSTRACT

Methods for treating mammals before, during and after cardiac arrest are disclosed. Pharmaceutical compositions comprising levosimendan useful for such treatment also are disclosed.

The present application claims priority to U.S. Provisional Application Ser. No. 60/575,765, filed on May 28, 2004, hereby incorporated in its entirety by reference.

### FIELD OF THE INVENTION

The present invention relates to methods for treating a mammal before, during and after cardiac arrest as well as pharmaceutical compositions suitable for use in said methods.

### BACKGROUND OF THE INVENTION

Cardiovascular disease continues to be the leading cause of death in the Western world. When a person suffers a cardiac arrest, whether inside a hospital or elsewhere, the survival rate is relatively low. Moreover, though the initial success of cardiopulmonary resuscitation is approximately 39% (range 13 to 59%), a majority of these victims die within 72 hours, primarily due to heart failure and/or recurrent ventricular fibrillation. Unfortunately, only 5% or one of 8 out-of-hospital successfully resuscitated patients survives hospitalization. Reversible myocardial dysfunction has been observed after successful resuscitation from cardiac arrest in experimental models (Tangetal., Crit. Care Med., 21:1046-1050 (1993); Tang et al., Circulation, 92:3089-3093 (1995); Gazmuri et al., Crit. Care Med., 24:992-1000 (1996); Kern et al., J. Am. Coll. Cardiol., 28:232-240 (1996)) and in human patients (Deantonio et al. Pacing Clin. Electrophysiol., 13:982-985 (1990)). This dysfunction peaks at 2 to 5 hours in a rat model and it is typically resolved within 72 hours (Kern et al., J. Am. Coll. Cardiol., 28:232-240 (1996)). In human victims, the impairment of myocardial contractile function may persist for intervals of one to two weeks (Deantonio et al. Pacing Clin. Electrophysiol., 13:982-985 (1990)). The phenomenon of reversible ventricular dysfunction after transient coronary occlusion is viewed as comparable to that described as “stunned” myocardium in settings of acute myocardial infarction (Braunwald et al., Circulation, 66(6):1146-9(1982)). This may explain, at least in part, the high fatality rate due to ventricular arrhythmias and heart failure within the initial 72 hours after successful resuscitation from cardiac arrest (Liberthson et al., N. Engl. J Med., 291(7):317-321 (1974)).

Typically, the responsiveness of a heart in cardiac arrest, to defibrillation and subsequent restoration or return of spontaneous circulation (ROSC) depends on the total time of ischemia from cardiac arrest to that of interventions including CPR and defibrillation. The longer the ischemia time and the longer the duration of ventricular fibrillation, the more difficult it becomes to engender a response to the Advanced Cardiac Life Support (ACLS) protocols including defibrillation. (ACLS guidelines, 1^{st} paragraph, p. I90; also MH Hayes, RA Berg, CW Otto Current Opinion Critical Care 2003; 9: 211-217). This is due to ischemia producing a higher defibrillation threshold time requiring more defibrillation attempts and/or greater defibrillation energies. Furthermore many of the agents recommended in the ACLS guidelines such as epinephrine, and other agents such as lidocaine, also raise defibrillation thresholds. The greater cumulative defibrillation energies and attempts produce greater myocardial injury and dysfunction, and impaired circulation and organ perfusion post-resuscitation. Such impaired or failed organ perfusion further contributes to the post-resuscitation syndrome (ACLS guidelines, p. I166) and poor recovery and outcomes for the cardiac arrest victim. Post-resuscitation myocardial dysfunction often produces myocardial electrical instability and recurrent arrhythmias, necessitating further defibrillation attempts and the potential for greater myocardial injury. (Gazmuri et al, Current Opinion Critical Care 2003; 9 199-204).

Other factors involved in the process of resuscitating (i.e. restoring ventilation and circulation) in a patient also may contribute to increased myocardial injury and dysfunction. For example, currently available agents such as dobutamine or norepinephrine or epinephrine may be used to treat myocardial stunning or dysfunction but can produce and/or exacerbate myocardial and organ ischemia, increase oxygen consumption and increase calcium flux into cells. In addition, other drugs with β receptor agonist activity (like epinephrine), which are used to treat cardiac arrest and/or post-resuscitation recovery, increase myocardial electrical instability and ectopic activity due to β receptor stimulation (Gazmuri, et al., supra) and also may produce increased oxygen consumption and calcium influx into cells via β receptor agonism. The use of β receptor antagonists to treat the effects of β receptor agonists to improve post-resuscitation recovery has been described (Gazmuri, et al., supra). However, β receptor antagonists are negative inotropes that may contribute to impairment of cardiac function during or after resuscitation. In addition, vasopressin is used to treat cardiac arrest by improving coronary perfusion pressure without the negative effects of β receptor agonism. However the vasoconstrictive effects of vasopressin have a greater duration in the postresuscitation period and compromise organ blood flow. Prolonged vasoconstriction also exacerbates myocardial dysfunction by increasing cardiac afterload.

Accordingly, there is a need in the art for methodologies and drugs that will protect the myocardium and other organs and tissues before, during and after cardiac arrest. More specifically, there is a need in the art for treatment methods that will improve time to ROSC, lower defibrillation thresholds, minimize or prevent myocardial dysfunction pre- or post-resuscitation, minimize or prevent reperfusion injury and/or improve survival rates of individuals who have suffered from cardiac arrest.

### SUMMARY OF THE INVENTION

The present invention generally relates to methods for treating a mammal before, during or after cardiac arrest as well as pharmaceutical compositions containing levosimendan that are suitable for use in these methods. In one embodiment, the invention provides a method for restoring spontaneous circulation in a mammal in cardiac arrest wherein the method comprises the steps of administering cardiopulmonary resuscitation (CPR) and defibrillation shocks to the mammal, the improvement comprising administering to the mammal a therapeutically effective amount of a levosimendan compound or a pharmaceutically acceptable salt thereof. Preferably, the levosimendan compound is levosimendan or a metabolite of levosimendan. Preferably, the step of administering the levosimendan compound occurs at the onset of administering CPR.

In a second embodiment, the invention provides a method for reducing the frequency of defibrillation shocks applied to a mammal in cardiac arrest, the method comprising the steps of administering a therapeutically effective amount of a levosimendan compound or a pharmaceutically acceptable salt thereof to the mammal prior to applying the defibrillation shocks; and applying the defibrillation shocks at a frequency sufficient to restore effective cardiac rhythm, wherein the frequency is reduced relative to the frequency established by a recognized standard of care protocol.

In an alternative embodiment, the invention provides a method for reducing the frequency of defibrillation shocks applied to a mammal in cardiac arrest, the method comprising the steps of:administering a therapeutically effective amount of a levosimendan compound or a pharmaceutically acceptable salt thereof to the mammal prior to applying said defibrillation shocks; and applying the defibrillation shocks at a frequency sufficient to restore effective cardiac rhythm, wherein the frequency is reduced relative to the frequency of defibrillation shocks applied to a similar mammal in cardiac arrest which has not been treated with the levosimendan compound.

In a third embodiment, the invention provides a method of reducing the energy of a defibrillation shock applied to a mammal in cardiac arrest, the method comprising the steps of administering a therapeutically effective amount of a levosimendan compound or a pharmaceutically acceptable salt thereof to the mammal prior to applying the defibrillation shock; and applying the defibrillation shock to the mammal at the energy sufficient to restore effective cardiac rhythm, wherein the energy is reduced relative to the energy established by a recognized standard of care protocol.

In an alternative embodiment, the invention provides a method of reducing the energy of a defibrillation shock applied to a mammal in cardiac arrest, the method comprising the steps of administering a therapeutically effective amount of a levosimendan compound or a pharmaceutically acceptable salt thereof to the mammal prior to applying the defibrillation shock; and applying the defibrillation shock to the mammal at the energy sufficient to restore effective cardiac rhythm, wherein the energy is reduced relative to the energy applied to a similar mammal in cardiac arrest which has not been treated with the levosimendan compound.

In a fourth embodiment, the invention provides a method of treating myocardial dysfunction in a mammal in need thereof during or after resuscitation from cardiac arrest, comprising the step of administering to the mammal a therapeutically effective amount of a levosimendan compound or a pharmaceutically acceptable salt thereof.

In a fifth embodiment, the invention provides a method for treating cardiac arrhythmia in a mammal in need thereof, wherein the method comprises the step of applying one or more defibrillation shocks to the mammal, the improvement comprising administering to the mammal, a therapeutically effective amount of a levosimendan compound or pharmaceutically acceptable salt thereof. Preferably, the administration of the levosimendan compound occurs after said applying one or more defibrillation shocks.

In a sixth embodiment, the invention provides a method for protecting organ function in a mammal subsequent to cardiac arrest, wherein the method comprises the step of restoring spontaneous circulation in the mammal, the improvement comprising administering to the mammal a therapeutically effective amount of a levosimendan compound or a pharmaceutically acceptable salt thereof. Preferably, the organ function is brain, renal or hepatic organ function.

In a seventh embodiment, the invention provides a method for preventing myocardial dysfunction in a mammal in need thereof prior to cardiac arrest or global ischemia comprising the step of administering to the mammal a therapeutically effective amount of a levosimendan compound or a pharmaceutically acceptable salt thereof.

In any or all of the aforementioned embodiments, the administration of the levosimendan compound comprises administering the compound as either a single dose administration or as a continuous infusion. Preferably, administration to the mammal is via a parenteral route and more preferably, by intravenous, endotracheal, intraarterial, transdermal or intracardiac administration.

In any or all of the aforementioned embodiments, a preferred mammal is a human. In addition, in any and all of the aforementioned embodiments, administration of the levosimendan compound to the mammal is in an amount of from about 0.01 to about 5.0 μg/kg/minute, preferably, in an amount of from about 0.05 to about 0.4 μg/kg/minute and more preferably, in an amount of from about 0.1 μg/kg/minute. Alternatively, administration of the levosimendan compound is in an amount of from about 0.06 to about 36 μg/kg.

In addition, in any or all of the aforementioned embodiments of the invention, the method further comprises the step of administering a therapeutically effective amount of an adrenergic receptor-blocking agent to the mammal. The adrenergic receptor-blocking agent may be a beta adrenergic receptor-blocking agent or an alpha adrenergic receptor-blocking agent. If a beta adrenergic receptor-blocking agent, the agent may be a beta- I adrenergic receptor-blocking agent or a beta-2 adrenergic receptor-blocking agent. Preferably, a beta adrenergic receptor-blocking agent is propanolol, metoprolol, esmolol or atenolol. Alternatively, if an alpha adrenergic receptor-blocking agent, the agent is an alpha-1 adrenergic receptor-blocking agent. A preferred agent, which has been characterized as either a beta or alpha adrenergic receptor-blocking agent is carvedilol.

### BRIEF DESCRIPTION OF THE DRAWINGS

FIG. 1 shows a graph measuring the cardiac index in ml/kg/min in rats treated with 0.4 μg/kg/min. of levosimendan (□), 0.3 μg/kg/min. of levosimendan (Δ), 2 μg/kg/min. of levosimendan (∘) and a placebo (♦).

FIG. 2 shows a graph measuring the mean arterial pressure in mmHg in rats treated with 0.4 μg/kg/min. of levosimendan (□), 0.3 μg/kg/min. of levosimendan (Δ), 2 μg/kg/min. of levosimendan (∘) and a placebo (♦).

FIG. 3 shows a graph measuring the heart rate in beats/minute in rats treated with 0.4 μg/kg/min. of levosimendan (□), 0.3 μg/kg/min. of levosimendan (Δ), 2 μg/kg/min. of levosimendan (∘) and a placebo (♦).

FIG. 4 shows a graph measuring the mean arterial pressure in mmHg for mice treated post-resuscitation with levosimendan (▪), dobutamine (∘) and a placebo (Δ).

FIG. 5 shows a graph measuring heart rate in beats/minute in rats treated post-resuscitation with levosimendan (▪), dobutamine (∘) and a placebo (Δ).

FIG. 6 shows a graph measuring cardiac index in ml/kg/min. in rats treated post-resuscitation with levosimendan (▪), dobutamine (∘) and a placebo (Δ).

FIG. 7 shows a graph measuring stroke volume in rats treated post-resuscitation with levosimendan (▪), dobutamine (∘) and a placebo (Δ).

FIG. 8 shows a graph measuring systemic vascular resistance in rats treated post-resuscitation with levosimendan (▪), dobutamine (∘) and a placebo (Δ).

FIG. 9 shows a graph measuring contractility (as reflected in the dP/dt40) in rats treated post-resuscitation with levosimendan (▪), dobutamine (∘) and a placebo (Δ).

FIG. 10 shows a graph measuring lusitropic or relaxation effect (as reflected in negative dP/dt40) in rats treated post-resuscitation, with levosimendan (▪), dobutamine (∘) and a placebo (Δ).

FIG. 11 shows a graph measuring the left ventricular diastolic (filling) pressures (LVDP) measured in mmHg in rats treated post-resuscitation, with levosimendan (▪), dobutamine (∘) and a placebo (Δ).

FIG. 12 is a chart showing duration of survival, in hours, resulting from control, dobutamine and levosimendan treatment.

FIG. 13 is a graph showing the effect of three interventions on postresuscitation heart rate (beats per minute), mean arterial pressure (mm Hg) and cardiac index (ml min⁻¹ kg⁻¹). Values represent mean values and standard deviation. BL=baseline; DF=defibrillation; PC=precordial compression; VF=ventricular fibrillation. *P<0.05, **P<0.01 vs saline placebo; ^{†}P<0.05 vs dobutamine

FIG. 14 is a graph showing values of dP/dt₄₀ (mm Hg sec⁻¹×10³), −dP/dt (mm Hg sec⁻¹×10³) and P_{LVD} (mmHg). BL=baseline; DF=defibrillation; PC=precordial compression; VF=ventricular fibrillationU.S. Pat. No. . *P<0.05; **P<0.01 vs saline placebo

FIG. 15 is a chart showing survival time at 72 hours. BL=baseline; DF=defibrillation; PC=precordial compression; VF=ventricular fibrillation. *P<0.05, **P<0.01 vs saline placebo; ^{†}P<0.05 vs dobutamine.

FIG. 16 is a graph showing the effect of three interventions on postresuscitation cardiac output (mL min⁻¹). Values represent mean values and standard deviation. BL=baseline; DF=defibrillation; PC=precordial compression; VF=ventricular fibrillation. *P<0.05, **P<0.01 vs saline placebo

FIG. 17 is a graph showing values of Ejection Fraction (EF, %). Values represent mean values and standard deviation. BL=baseline; DF=defibrillation; PC=precordial compression; VF=ventricular fibrillation. *P<0.05; **P<0.01 vs saline placebo, ^{†}P<0.05, ^{††}P<001 vs dobutamine

FIG. 18 is a graph showing values of FAC (%). Values represent means and bars represent ±S.D. BL=Baseline. VF=Ventricular fibrillation. PC=Precordial compression. DF=Defibrillation. *P<0.05; **P<0.01 vs saline placebo, ^{†}P<0.05, vs dobutamine

FIG. 19 is a graph showing values of PO₂ difference between artery and great cardiac vein blood (Pa-vO₂). Values represent means and bars represent ±S.D. BL=Baseline. VF=Ventricular fibrillation. PC=Precordial compression. DF=Defibrillation. *P<0.05 vs saline placebo

FIG. 20 is a chart showing EF and Pa-vO₂ percentage of BL level at 240 minutes after resuscitation.

FIG. 21 is a graph showing values of lactate of great cardiac vein blood.

FIG. 22 is a is a graph showing increases in cardiac index (CI), contractility (dP/dt40), and mean arterial pressure (MAP) after levosimendan (solid circles) in comparison with saline placebo (open squares). Values represent means and bars represent ±S.D. BL=Baseline. VF=Ventricular fibrillation. PC=Precordial compression. DF=Defibrillation.

FIG. 23 is a is a graph showing decreased left ventricular diastolic pressure (LVDP) and increases in negative dP/dt consistent with improved diastolic ventricular function increases in end-tidal CO₂ (ETCO₂) are consistent with increases in cardiac output. Levosimendan (solid circles), saline placebo (open squares). Values represent means and bars represent ±S.D. BL=Baseline. VF=Ventricular fibrillation. PC=Precordial compression. DF=Defibrillation.

FIG. 24 is a graph showing decreased peripheral arterial resistance (PAR) after levosimendan (solid circles) vs saline placebo (open squares). Values represent means and bars represent ±S.D. BL=Baseline. VF=Ventricular fibrillation. PC=Precordial compression. DF=Defibrillation.

FIG. 25 is a graph showing the experimental procedure for carrying out the study. VF=Ventricular fibrillation. DF=defibrillation.

FIG. 26 is a chart showing significantly improved defibrillating shocks, number of PVB, and ST-T elevations in propranolol group. Values are shown as mean±S.D.

FIG. 27 is a graph showing significantly greater FAC and EF in levosimendan+propranolol and propranolol groups compared to control. Values are shown as mean ±S.D.

### DETAILED DESCRIPTION OF THE INVENTION

All abstracts, references, patents and published patent applications referred to herein are hereby incorporated by reference in their entirety.

As used herein, the phrase “adrenergic receptor-blocking agent” refers to any agent which acts to block an adrenergic receptor. In the context of the present invention, such agents therefore include recognized adrenergic receptor-blocking agents such as propanolol, metoprolol, carvedilol, as well as other compounds which have this blocking activity.

As used herein, the phrase “cardiac arrhythmia” refers to an abnormal cardiac rate or rhythm. The condition may be caused by a defect in the node to maintain its pacemaker function, or by a failure of the electrical conduction system. Examples of arrhythmia include, but are not limited to bradycardia, tachycardia (such as supraventricular tachycardia and ventricular tachycardia), ventricular fibrillation and extrasystole. “Treating cardiac arrhythmia” refers to alleviating or reversing the condition of cardiac arrhythmia.

As used herein, the term “bradycardia” refers to a circulatory condition in which the heart contracts steadily but at a rate of less than 60 contractions a minute.

As used herein, the phrase “cardiac arrest” refers to a cessation of cardiac output and effective circulation. Cardiac arrest is typically precipitated by cardiac arrhythmias such as ventricular tachycardia and ventricular fibrillation (or both) or bradycardia. Cardiac arrest may result from heart disease or heart attack or from other factors such as respiratory arrest, electrocution, drowning, choking and trauma. When cardiac arrest occurs, delivery of oxygen and removal of carbon dioxide stop, tissue cell metabolism becomes anaerobic, and metabolic and respiratory acidosis ensue. Immediate initiation of cardiopulmonary resuscitation is required to prevent heart, lung, kidney and brain damage. Brain death and permanent death start to occur within 4-6 minutes of arrest.

As used herein, the phrase “cardiopulmonary resuscitation” or “CPR” refers to a process of applying mouth-to-mouth ventilation and chest compressions (typically, by an individual and without without the aid of a device) to an individual in need thereof. Standard of care guidelines for applying CPR are well established in the art (see e.g. guidelines on Advanced Cardiac Life Support (ACLS) of the American Heart Association (AHA) /International Liaison Committee on Resuscitation (ILCOR)). (See, e.g. Supplement to Circulation, Vol. 102(8), Aug. 22, 2000)

As used herein, the phrase “congestive heart failure” refers to an abnormal condition of the heart characterized by an impaired ability to pump sufficient blood the body's other organs. Congestive heart failure may result from any number of conditions, including coronary artery disease, myocardial infarction, endocarditis, myocarditis or cardiomyopathy. Failure of the ventricle to eject blood results in volume overload, chamber dilatation, and elevated intracardiac pressure. Retrograde transmission of increased hydrostatic pressure from the left heart causes pulmonary congestion; elevated right heart pressure causes systemic venous congestion and peripheral edema.

As used herein, the term “defibrillation” refers to the arrest or cessation of fibrillation of the cardiac muscle (atrial or ventricular) with restoration of effective cardiac rhythm. Typically, defibrillation is achieved by with the aid of a device (e.g. a defibrillator) that delivers an electrical shock.

As used herein, the phrase “effective cardiac rhythm” is cardiac rhythm which achieves the desired therapeutic result, such as for example, stabilization of the individual and/or survival.

As used herein, the term “extrasystole” refers to an abnormal cardiac contraction that results from depolarization by an ectopic impulse.

As used herein, the term “ischemia” refers to a condition in which blood flow is restricted to a part of the body. Ischemia may result from mechanical obstruction (for example, arterial narrowing) of the blood supply. “Regional ischemia” refers to a condition in which a portion of the organ receives restricted blood flow. “Global ischemia” refers to a condition in which the entire organ receives restricted blood flow.

As used herein, the term “levosimendan compound” refers to any racemic mixture or enantiomer of levosimendan or a racemic mixture or enantiomer of the metabolite of levosimendan. The term “levosimendan” specifically refers to the (−)-enantiomer of [4-(1,4,5,6-tetrahydro4-methyl-6-oxo-3-pyridazinyl)phenyl]hydrazono]propanedinitrile.

As used herein, the term “mammal” refers to any vertebrate of the class Mammalia, having the body more or less covered with hair, nourishing the young with milk from the mammary glands, and, with the exception of the egg-laying monotremes, giving birth to live young. Examples of mammals include, but are not limited to, mice, rats, cats, dogs, pigs, monkeys and human beings. The preferred mammal is a human being.

As used herein, the phrase “myocardial dysfunction” refers to a condition of the heart characterized by reduced cardiac output, decreased cardiac contractility and decreased arterial pressure with increases in left ventricular filling pressures that accompany, arise from or are caused by cardiac arrest or the treatment(s) used to treat cardiac arrest. “Treatment of myocardial dysfunction” or “improving myocardial dysfunction” refers to easing, attenuating, reversing or alleviating the condition of myocardial dysfunction. Myocardial function/dysfunction is measured using instrumentation and means well known to those of ordinary skill in the art.

As used herein, the phrase “pharmaceutically acceptable salt” refers to the salt forms of an active ingredient, such as levosimendan, that is physiologically suitable for pharmaceutical use.

As used herein, the phrase “protecting organ function” refers to restoring effective organ function, maintaining effective organ function or preventing further deterioration of organ function in a mammal after cardiac arrest.

As used herein, the phrase “recognized standard of care protocol” refers to a series of instructional guidelines that are accepted by practitioners in the field as a means of treating a particular condition. By way of example, the guidelines established by the AHA/LIROC for administering CPR and defibrillation to individuals suffering from cardiac arrest is a recognized standard of care protocol As used herein, the phrase “restoring spontaneous circulation”, “return of spontaneous circulation” or “ROSC” refers to a return or re-initiation of blood circulation of an individual's own accord. Additional supportive measures may or may not be require to assist an individual in maintaining spontaneous circulation.

As used herein, the term “tachycardia” refers to a condition of the heart in which the heart contracts at a rate greater than 100 beats per minute.

As used herein, the phrase “ventricular fibrillation” refers to a condition of the heart that is characterized by a lack of organized electric impulse, conduction, and ventricular contraction.

The invention provides an improved method for treating a mammal suffering from a particular condition of impaired myocardial function. More specifically, the present invention provides a method for treating a mammal suffering from global cardiac ischemia or any arrhythmia preceding such ischemia. Even more specifically, the methods of the invention comprise administering to a mammal experiencing the conditions described above and in need of such treatment, a therapeutically effective amount of a levosimendan compound or pharmaceutically acceptable salt thereof.

In one aspect, the present invention relates to an improved method of restoring spontaneous circulation in a mammal in cardiac arrest. Specifically, the improvement comprises administering a therapeutically effective amount of a levosimendan compound or a pharmaceutically acceptable salt thereof to a mammal in cardiac arrest and in need of such treatment, wherein the mammal is subjected to or will be subjected to cardiopulmonary resuscitation (CPR) and defibrillation shocks to restore spontaneous circulation. The American Heart Association (AHA), in conjunction with the International Liaison Committee on Resuscitation (ILROC), has established guidelines for resuscitating individuals experiencing cardiac arrest, which include procedures for restoring spontaneous circulation. These guidelines constitute a standard of care protocol recognized by emergency medical system (EMS) personnel (e.g. paramedics) and hospital staff for treating individuals in cardiac arrest and are administered routinely by these and other health care providers in both a hospital and non-hospital setting. However, it also is understood by those skilled in the art that the guidelines apply generally to all individuals in need of such treatment but that the actual treatment performed may vary from individual to individual depending on need.

The step of administering a levosimendan compound or a pharmaceutically acceptable salt thereof to a mammal can be performed just prior to the time that the mammal is expected to experience a cardiac arrest or at any time during the time that the mammal is in actual cardiac arrest or after a cardiac arrest event. Furthermore, the step of administering a levosimendan compound may be accomplished either by administeration of a levosimendan compound as a single or bolus dose or by continuous infusion. Methods for determining when a mammal is likely to experience a heart attack or is in actual cardiac arrest are well known and within the skill of ordinary practitioner in the art and include, but are not limited to, the use of an electrocardiogram (ECG) and laboratory tests for creatine kinase-MB, myoglobin and troponin I.

In another embodiment, the present invention relates to the discovery that the administration of a therapeutically effective amount of levosimendan or a pharmaceutically acceptable salt thereof to a mammal prior to defibrillation therapy can (1) reduce the number of times that defibrillation therapy must be repeated on a mammal experiencing ventricular fibrillation in order to reinitiate a hemodynamically effective cardiac function; and/or (2) reduce the amount of energy (i.e., current) applied during defibrillation therapy to reinitiate a hemodynamically effective cardiac function in a mammal experiencing ventricular fibrillation.

In one aspect, the method of the present invention comprises the steps of administering to a mammal prior to or in cardiac arrest, a therapeutically effective amount of a levosimendan compound or a pharmaceutically acceptable salt thereof prior to applying one or more defibrillation shocks and applying the defibrillation shock(s) at a frequency (i.e. for a number of times) sufficient to restore effective cardiac rhythm, wherein the frequency is reduced relative to the frequency established by a recognized standard of care protocol. As mentioned supra, recognized standard of care protocols have been established by, for example, the AHA/ILROC for defibrillating an individual in cardiac arrest. Such individual may or may not need CPR. Preferably, the number of defibrillation shocks is reduced by 50%, more preferably by 60%, more preferably by 70%, more preferably by 80%, even more preferably by 90% and even more preferably by 100%.

In an alternative aspect, the invention provides a method for reducing the frequency of defibrillation shocks applied to a mammal in cardiac arrest comprising the steps of administering a therapeutically effective amount of a levosimendan compound or a pharmaceutically acceptable salt thereof to the mammal prior to applying one or more defibrillation shocks and applying the defibrillation shock(s) at a frequency sufficient to restore effective cardiac rhythm, wherein the frequency is reduced relative to the frequency of defibrillation shocks applied to a similar mammal in cardiac arrest which has not been treated with the levosimendan compound. Preferably, the number of defibrillation shocks is reduced by 50%, more preferably by 60%, more preferably by 70%, more preferably by 80%, even more preferably by 90% and even more preferably by 100%.

In yet another aspect, the invention provides a method of reducing the energy of a defibrillation shock applied to a mammal in cardiac arrest comprising the steps of administering a therapeutically effective amount of a levosimendan compound or a pharmaceutically acceptable salt thereof to the mammal prior to applying one or more defibrillation shocks and applying the defibrillation shock(s) to the mammal at an energy sufficient to restore effective cardiac rhythm, wherein the energy is reduced relative to the energy of a defibrillation shock established by a recognized standard of care protocol. Preferably, the energy of defibrillation shocks is reduced by 50%, more preferably by 60%, more preferably by 70%, more preferably by 80%, even more preferably by 90% and even more preferably by 100%.

In yet another alternative aspect, the invention provides a method of reducing the energy of a defibrillation shock applied to a mammal in cardiac arrest comprising the steps of administering a therapeutically effective amount of a levosimendan compound or a pharmaceutically acceptable salt thereof to the mammal prior to applying one or more defibrillation shocks and applying the defibrillation shock(s) to the mammal at an energy sufficient to restore effective cardiac rhythm, wherein the energy is reduced relative to the energy of defibrillation applied to a similar mammal in cardiac arrest which has not been treated with said levosimendan compound. Preferably, the energy of defibrillation shocks is reduced by 50%, more preferably by 60%, more preferably by 70%, more preferably by 80%, even more preferably by 90% and even more preferably by 100%.

In any of the embodiments and/or aspects disclosed herein, defibrillation therapy may be provided by a defibrillator, which delivers an electrical shock to the chest area of a mammal or directly to the heart itself in an attempt to reinitiate a hemodynamically effective cardiac function in a subject experiencing ventricular fibrillation. Defibrillation electrodes are preferably located on opposite sides of the heart (such as on the left lateral and right lateral ventricular epicardium), such that as much cardiac muscle mass as possible is located within the direct current path of the defibrillating shock. Typically, a defibrillator delivers between from about 200 joules to about 400 joules of energy to the subject. The key to a successful defibrillation is to have enough energy (i.e., current) delivered to the heart to stop ventricular fibrillation or other arrhythmia. The energy should not be high enough to injure (such as to burn or cause memory loss) the subject being treated. Generally, after the first attempt at defibrillation, the energy (current) applied in each subsequent defibrillation attempt is increased, thereby increasing the risk of injury to the subject. While defibrillation therapy is a very important medical tool, each defibrillating shock applied increases the risk of injury to the subject being treated.

Various types of defibrillators are known in the art. Specifically, defibrillators can be external (such as a manual defibrillator or an automatic external defibrillator) or can be internal (such as an implantable cardioverter defibrillator). Typically, implanted defibrillators monitor the subject's heart activity and automatically supply electrotherapeutic pulses to the subject's heart whenever necessary. The step of providing defibrillation therapy to a mammal in the method of the present invention can occur at any time during the treatment of the mammal, such as, but not limited to, prior to, during or after cardiac arrest. Preferably, providing defibrillation therapy occurs at the onset of cardiac arrest. Additionally, the defibrillation may occur prior to, during or after the administration of a levosimendan compound or a pharmaceutically acceptable salt thereof according to the invention.

In yet a further embodiment, the present invention provides an improved method of treating a mammal exhibiting a cardiac arrhythmia, such as, but not limited to, supraventricular tachycardia, ventricular tachycardia, ventricular fibrillation or extrasystole. Specifically, the improvement comprises administering a therapeutically effective amount of a levosimendan compound or a pharmaceutically acceptable salt thereof to a mammal exhibiting a cardiac arrhythmia and in need of such treatment, wherein the animal is subjected to one or more defibrillation shocks.

The step of administering a levosimendan compound or a pharmaceutically acceptable salt thereof to a mammal in need of treatment can be made at any time during which the mammal is exhibiting a cardiac arrhythmia. Methods for determining a cardiac arrhythmia are well within the skill of ordinary practitioners in the art and include the use of an electrocardiogram.

In yet a further embodiment, the present invention relates to a method of preventing myocardial dysfunction in a mammal in need thereof prior to cardiac arrest or global ischemia comprising the step of administering to the mammal a therapeutically effective amount of a levosimendan compound or a pharmaceutically acceptable salt thereof. Such “preconditioning” protects the myocardium from ischemic damage that would occur during cardiac arrest. The step of administering a levosimendan compound or pharmaceutically acceptable salt thereof to a mammal in need of such treatment can be made, for example, prior to cardiac surgery.

In yet a further embodiment, the present invention relates to a method of treating myocardial dysfunction in a mammal resuscitated after suffering cardiac arrest. The method involves the step of administering a therapeutically effective amount of levosimendan or a pharmaceutically acceptable salt thereof to a mammal that has been resuscitated after a cardiac arrest and in need of such treatment. Specifically, the inventors have discovered that a levosimendan compound or a pharmaceutically acceptable salt thereof can be used to improve myocardial function as well as increase the length of a mammal's survival post-resuscitation. More specifically, the inventors have discovered that a levosimendan compound or a pharmaceutically acceptable salt thereof improves the cardiac function of the heart, lowers ventricular filling pressures and provides for a greater inotropic effect, when administered to a mammal after spontaneous circulation has been restored.

The step of administering levosimendan or a pharmaceutically acceptable salt thereof to a mammal in need of treatment can be made at any time after a subject has restored spontaneous circulation after a cardiac arrest and is exhibiting myocardial dysfunction. Methods for determining myocardial dysfunction are well known in the art and include the use of an electrocardiogram.

In another embodiment, the invention provides an improved method for protecting organ function in a mammal in need thereof. Specifically, the improvement comprises administering a therapeutically effective amount of a levosimendan compound or a pharmaceutically acceptable salt thereof to a mammal in need of such treatment, wherein the mammal has restored spontaneous circulation. Notwithstanding the need to restore spontaneous circulation, the reperfusion of body organs and tissues resulting from ROSC may cause a condition in the mammal known as “reperfusion injury”. “Reperfusion injury” refers to a spectrum of reperfusion-associated pathologies, manifested by, among other conditions, myocardial stunning, microvascular and endothelial injury and irreversible cell damage or necrosis (Subodh Verma, et al, Fundamentals of Reperfusion Injry for the Clinical Cardiologist, Circulation, Vol. 105:2332-2336 (2002). Mediators of reperfusion injury may include oxygen free radicals, intracellular calcium overload, endothelial and microvascular dysfunctio and altered myocardial metabolism (S. Verma et al., supra). Accordingly, in one aspect, the present invention provides a method for protecting organ function from the effects of reperfusion injury. The present method may protect any organ, but preferably protects brain, kidney, liver and heart tissue. As those of ordinary skill in the art will understand, the degree of protection afforded by the present invention will vary depending on the initial severity of organ damage. The step of administering a levosimendan compound or a pharmaceutically acceptable salt thereof to a mammal in need of organ protection can be made at any time prior to or after restoration or return of spontaneous circulation.

Methods for determining organ dysfunction/function are well known to those of ordinary skill in the art and include any means for measuring organ function or injury. For example, organ dysfunction/function may be measured by assessing levels of enzymatic or other markers of organ viability including, but not limited to cardiac troponin I (for cardiac tissue), creatinine or BUN (for renal tissue) serum AST and ALT (for hepatic tissue) and the like. Other means for measuring organ viability include electroencephalogram for brain tissue, electrocardiogram for heart tissue and the like.

In any of the embodiments and/or aspects described herein, the step of administering a levosimendan compound, the compound may be either a racemic mixture of levosimendan comprising both the (−) and (+) forms of [4-(1,4,5,6-tetrahydro4-methyl-6-oxo-3-pyridazinyl)phenyl]hydrazono]propanedinitrile or the (−)-enantiomer alone (e.g. (−)-[4-(1,4,5,6-tetrahydro-4-methyl-6-oxo-3 pyridazinyl)phenyl]hydrazono]propanedini-trile) or the racemic metabolite (N-[4-(1,4,5,6-tetrahydro4-methyl-6-oxo-3-pyridazinyl)phenyl]acetamide) or enantiomer metabolite ([R]- N-[4-(1,4,5,6-tetrahydro-4-methyl-6-oxo-3-pyridazinyl)phenyl]acetamide). A preferred levosimendan compound is. (−)-[4-(1,4,5,6-tetrahydro-4-methyl-6-oxo-3 pyridazinyl)phenyl]hydrazono]propanedinitrile. Methods for making the racemic mixture of levosimendan are described in U.S. Pat. No. 5,019,575, published May 28, 1991 and in EP Patent No. EP 0 383 449, published Sep. 6, 1995. Methods for making the (−)-enantiomer of [4-(1,4,5,6-tetrahydro-4-methyl-6-oxo-3-pyridazinyl)phenyl]hydrazono]propanedinitrile (i.e. levosimendan) are described U.S. Pat. No. 5,424,428, published Jun. 13, 1995 and in EP 0 565 546, published Mar. 8, 1995. Methods for preparing the racemic mixture of the metabolite of levosimendan are described in U.S. Pat. Nos. 3,746,712 and 4,397,854, published on Jul. 17, 1973 and Aug. 9, 1983, respectively. Methods for preparing the [R]-enantiomer of the metabolite are described in U.S. Pat. No. 5,905,078, published May 18, 1999 and U.S. Pat. No. RE 38,102 E, published Apr. 29, 2003 and EP 1 087 769, published Mar.10, 2004.

Furthermore, in any of the embodiments and/or aspects disclosed herein, other compounds also can be administered advantageously to the mammal just prior to or during a cardiac arrest. These compounds can be administered to the mammal before, after, or concurrently with the administration of the levosimendan compound or pharmaceutically acceptable salt thereof according to the invention. For example, a patient who has been treated with an adrenergic blocking agent and suffers a cardiac arrest episode may then be treated with a levosimendan compound. Examples of compounds that can be administered include adrenergic receptor-blocking agents, antithrombic agents, vasodilators and analgesics. Adrenergic receptor-blocking agents that can be administered include beta adrenergic receptor-blocking agents (such as, beta-1 adrenergic receptor-blocking agents or beta-2 adrenergic receptor-blocking agents) and alpha adrenergic receptor-blocking agents, such as alpha-1 adrenergic receptor-blocking agents. Examples of beta-adrenergic receptor-blocking agents that can be administered include, but are not limited to, atenolol, metoprolol, esmolol and propanolol and carvedilol. Examples of alpha adrenergic receptor-blocking agents include, but are not limited to, carvedilol. An example of an antithrombic agent that can be administered includes, but is not limited to, aspirin. An example of a vasodilator that can be administered, includes, but is not limited to, nitroglycerin. An example of an analgesic that can be administered, includes, but is not limited to, morphine sulfate. Generally, a therapeutically effective amount of any of the above-described compounds is administered to the mammal in need of treatment thereof and the actual amount to be administered will depend upon the condition to be treated, the route of administration, age, weight and the condition of the subject, and can be readily determined by the ordinary skilled physician.

According to the present invention, a levosimendan compound or a pharmaceutically acceptable salt thereof can be administered to a mammal in need of treatment through a variety of different routes known in the art, including enteral administration, such as through oral and rectal routes, or parenteral administration, such as through subcutaneous, intramuscular, intraperitoneal, sublingual, intravenous, endotracheal, intraarterial, transdermal or intracardiac routes. Exigency of circumstances surrounding treatment of the mammal may suggest a preferred route of administration, e.g., intracardiac injection.

As used herein, the term “therapeutically effective amount” or “pharmaceutically effective amount” means an amount of a levosimendan compound effective, at dosages and for periods of time necessary, to achieve the desired therapeutic result. A therapeutically effective amount of a levosimendan compound or a pharmaceutically acceptable salt thereof to be administered to a mammal will depend upon the condition to be treated, the route of administration, age, weight and the condition of the subject and is well within the skill of the ordinary physician. Generally, the levosimendan compound or pharmaceutically acceptable salt thereof can be administered in the amount of from about 0.01 to about 5.0 μg/kg/minute, preferably in the amount from about 0.5 to about 0.4 μg/kg/minute, and most preferably in the amount of about 0.1 μg/kg/minute. Depending upon the nature of the condition of the mammal, the levosimendan or a pharmaceutically acceptable salt thereof may be continuously administered from the time just prior to or during cardiac arrest until the time that the therapeutic effect is achieved. A bolus injection can be given or the injection can be followed by continuous administration, as described above.

In another embodiment, the present invention relates to a pharmaceutical formulation for treating cardiac arrest in a mammal. The pharmaceutical formulation of the present invention contains a therapeutically effective amount of a levosimendan compound or a pharmaceutically acceptable salt thereof and a pharmaceutically acceptable carrier. The pharmaceutical formulation of the present invention, when administered to a mammal in need of treatment, is sufficient to restore spontaneous circulation in said mammal, when the formulation is administered in conjunction with the administration of CPR and defibrillation. In another embodiment, the pharmaceutical formulation of the present invention, when administered to a mammal in need of treatment, is sufficient to reduce the frequency or energy of defibrillation shocks when administered in conjunction with defibrillation. In another embodiment, the pharmaceutical formulation of the present invention, when administered to a mammal in need of treatment, is sufficient to treat cardiac arrhythmias when administered in conjunction with defibrillation. In another embodiment, the pharmaceutical formulation, when administered to a mammal in need thereof, is sufficient to protect organ function when administered after resuscitation from cardiac arrest. The levosimendan compound or pharmaceutically acceptable salt thereof can be used in the pharmaceutical formulation in any form, but is preferably freeze-dried. Pharmaceutical formulations according to the invention can include other suitable excipients, carriers, or other compounds as necessary or desired.

The pharmaceutical formulation according to the invention may be prepared by mixing the active ingredient (such as, for example, levosimendan and any other compounds such as, but not limited to an adrenergic receptor-blocking agent) having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. Preferably, the pharmaceutical formulation of the present invention is substantially free of water. Acceptable carriers, excipients or stabilizers are nontoxic to recipients at the dosages and concentrations employed, and include buffers such as phosphate, citrate and other organic acids; antioxidants including ascorbic acid; low molecular weight (less than about 10 residues) polypeptides; proteins, such as serum albumin, gelatin or immunoglobulins; hydrophilic polymers such as polyvinylpyrrolidone, amino acids such as glycine, glutamine, asparagine, arginine or lysine; monosaccharides, disaccharides and other carbohydrates including glucose, mannose, or dextrins; chelating agents such as EDTA; sugar alcohols such as mannitol or sorbitol; salt-forming counterions such as sodium; and/or nonionic surfactants such as TWEEN™, PLURONICS™ or PEG.

Dosages and desired drug concentrations of pharmaceutical formulation of the present invention depend upon the condition to be treated, the route of administration, age, weight and the condition of the subject and are well within the skill of the ordinary physician. Additionally, animal experiments provide reliable guidance for the determination of effective doses for human therapy.

The present invention is illustrated by way of the foregoing description and examples. The foregoing description is intended as a non-limiting illustration, since many variations will become apparent to those skilled in the art in view thereof.

Changes can be made to the composition, operation and arrangement of the method of the present invention described herein without departing from the concept and scope of the invention.

### EXAMPLE 1

### Use of Levosimendan For Treating Myocardial Dysfunction in a Mammal Resuscitated After Suffering Cardiac Arrest

All animals received humane care in compliance with the Principles of Laboratory Animal Care formulated by the National Society for Medical Research and the Guide for the Care and Use of Laboratory Animals prepared by the Institute of Laboratory Animal Resources and Published by the National Institutes of Health (NIH publication 86-32, revised 1985).

Methods: Male Sprague-Dawley rats weighing 500-550 g were fasted overnight except for free access to water. The animals were anesthetized by intraperitoneal injection of pentobarbital (45 mg/kg). Additional doses (10 mg/kg) were administrated at intervals of approximately one hour, or as required to maintain anesthesia, except that no anesthetic agents were administered for 30 minutes before induction of cardiac arrest. The trachea was orally intubated with a 14 g cannula mounted on a blunt needle with a 145° angled tip according to the methods of Stark (Stark et al., J Appl. Physiol. Resp. Environ. Exercise Physiol., 51(5): 1355-1356 (1981)). Procedures for vascular catheterization, hemodynamic measurements, blood sampling, monitoring of ETCO₂, induction of VF and precordial compression were conducted as described in Von Planta I et al., J. Appl. Physiol., 65(6): 2641-2647 (1988).

A polyethylene catheter (PE 50, Becton-Dickinson) was advanced into the left ventricle from the surgically exposed right carotid artery for measurement of left ventricular pressure and both dP/dt₄₀ and negative dP/dtₘₐₓ. A thermocouple microprobe, 10 cm in length and 0.5 mm in diameter, was inserted into the right femoral artery, advanced to the aortic valve and then withdrawn to the more distal ascending aorta. Blood temperature was measured with this sensor. For cardiac output measurements, 0.2 ml of isotonic saline indicator at room temperature was injected into the right atrium through a catheter advanced from the left jugular vein. Duplicate thermodilution curves were obtained and recorded and the cardiac output was computed with a cardiac output computer system (Model CO100, ICCM, Palm Springs, Calif.).

Ventricular fibrillation (“VF”) was induced through a guide wire advanced from the right jugular vein into the right ventricle. A progressive increase in 60 Hz current to a maximum of 2 mA was delivered to the right ventricular endocardium, and current flow was continued for 3 minutes such as to prevent spontaneous defibrillation. Mechanical ventilation was stopped after onset of VF. After onset of VF, VF was untreated for 6 minutes and CPR<including ventilation and precordial compression with a pneumatically driven mechanical chest compressor, were completed. These procedures were as described in Von Planta et al., J. Appl. Physiol., 65(6): 2641-2647 (1988) and have been extensively described in the art (See, Tang et al., Circulation, 92: 3089-3093 (1995); Sun et al., J. Pharm. Exp. Ther., 291: 773-777 (1999)). Coincident with start of precordial compression, the animal was mechanically ventilated. Tidal volume was established at 0.65 ml/100 g animal weight, at a frequency of 100/min, and with an FiO₂ of 1.0. Precordial compression was maintained at a rate of 200/min and synchronized to provide a compression/ventilation ratio of 2:1 with equal compression-relaxation duration. Depth of compression was initially adjusted such as to secure a coronary perfusion pressure (CPP) of 18-22 mm Hg. This typically yielded an end-tidal PCO₂ of 8-12 mm Hg (See, Von Planta et al., J. Appl. Physiol., 65(6): 2641-2647 (1988)). A catheter was advanced into the left femoral artery for measurement of arterial pressure and blood gas. Another catheter was advanced into the left femoral vein for measurement of blood gases. Resuscitation was attempted with up to 3, two-joule countershocks. Restoration of spontaneous circulation was defined as the return of supraventricular rhythm with a mean aortic pressure of 60 mm Hg for a minimum of 5 min. In Group 1, animals were randomized by the sealed envelope method to one of three regimens, immediately after VF had been induced. A bolus dose of levosimendan (12 μg/kg) was followed by a continuous infusion of 0.3 μg/kg/min. In Group 2, a continuous infusion dose of dobutamine (3 μg/kg/min) was begun into the right atrium in the comparison group. For placebo in Group 3, an equivalent volume of levosimendan diluent was initially infused as a bolus followed by continuous infusion of volumes equivalent to those of both levosimendan and dobutamine. Infusions were continued for a total of 240 minutes post-resuscitation (PR). Mechanical ventilation was continued with 100% inspired oxygen for the entirety of the 4-hour post-resuscitation interval. Animals were allowed to recover from anesthesia and all catheters, including the endotracheal tube, were removed at the end of 4 hours. Animals were then returned to their cages. After autopsy, tissues (heart, liver, kidneys) were sampled and preserved in formalin for storage at room temperature.

The independent variable was levosimendan. The dependent variables were post-resuscitation myocardial function and duration of survival. The primary outcome variables for each experiment, including hemodynamic and metabolic measurements have previously proven to be appropriate for parametric analyses. Prior experience indicated normal distribution with homogeneity of variance. Accordingly, analysis of variance and analysis of covariance were the primary methods of data analysis. For measurements between groups, ANOVA and Scheffe's multi-comparison techniques were employed.

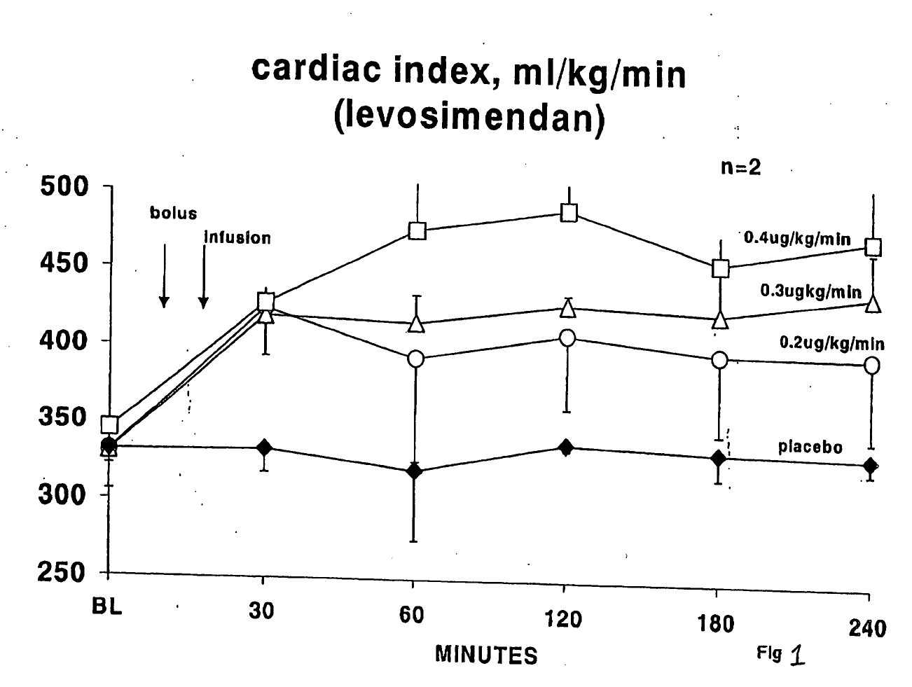

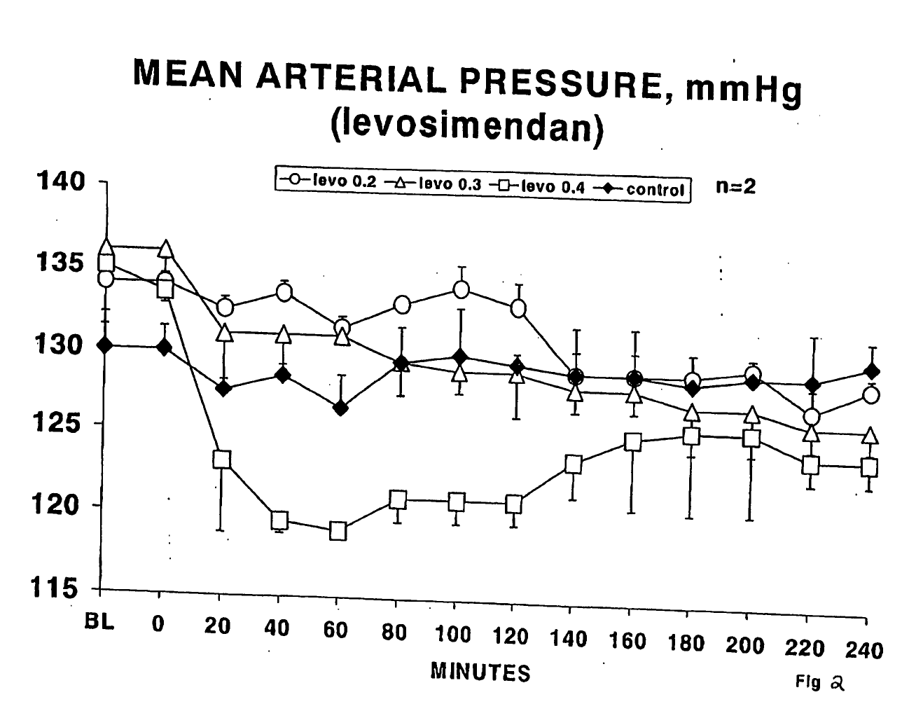

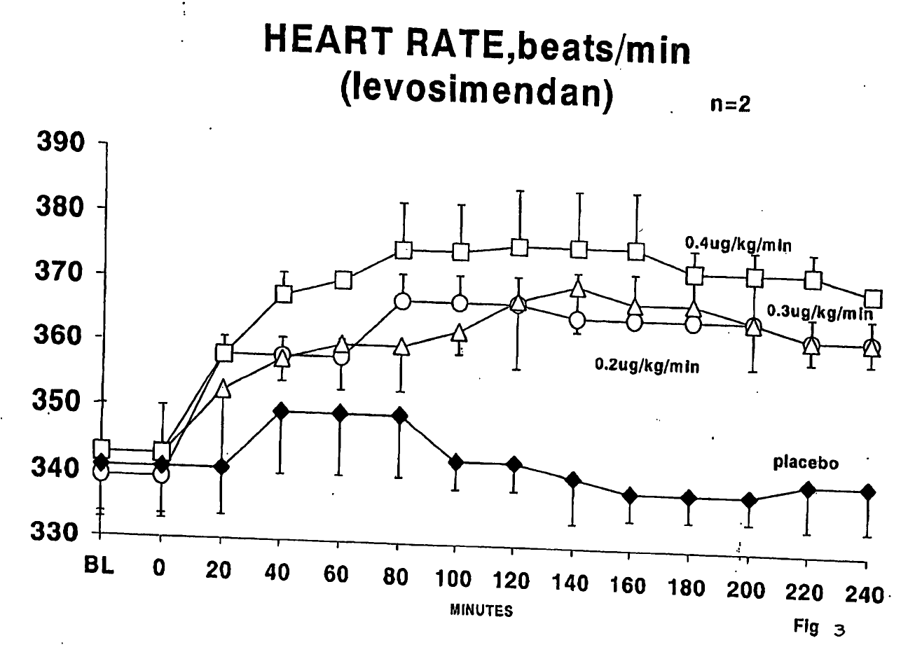

Feasibility Studies and Dose Titration: The results of dose titration are shown in FIGS. 1, 2 and 3. A dose of 12 μg of levosimendan followed by 0.3 μg/kg/min produced increases in cardiac output with declines in arterial pressure which were comparable to those produced by 3 μg/kg/min of dobutamine. Higher doses of levosimendan produced decreases in arterial pressure and increases in heart rate, even though there were additional increases in cardiac output.

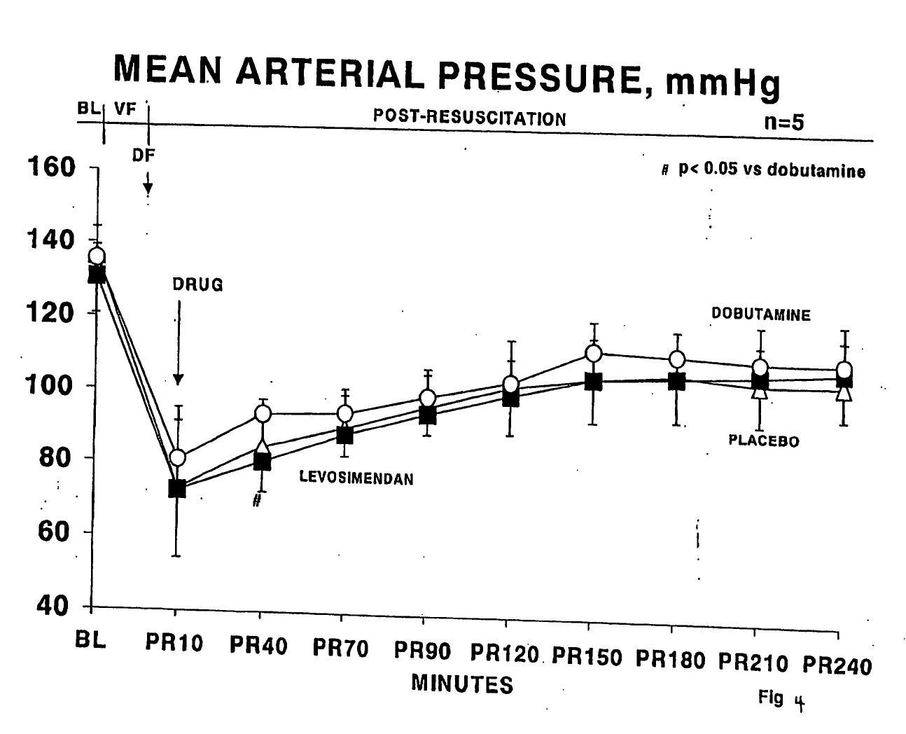

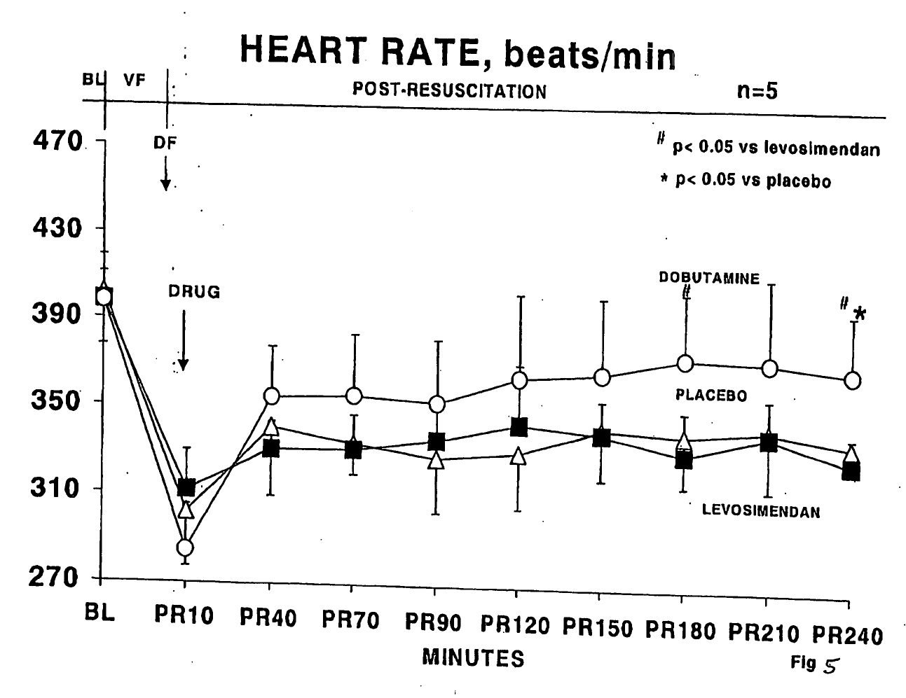

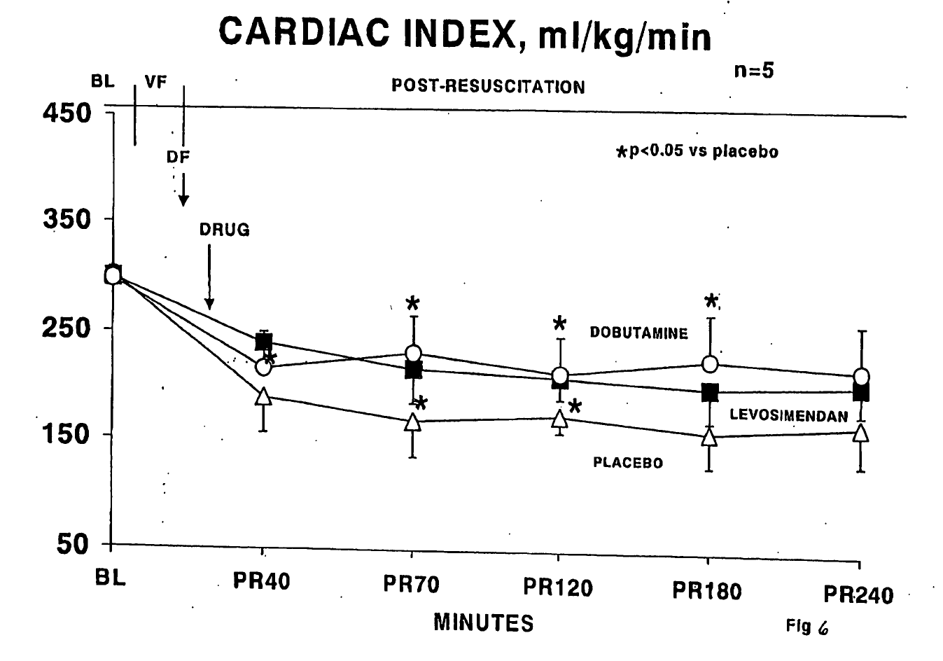

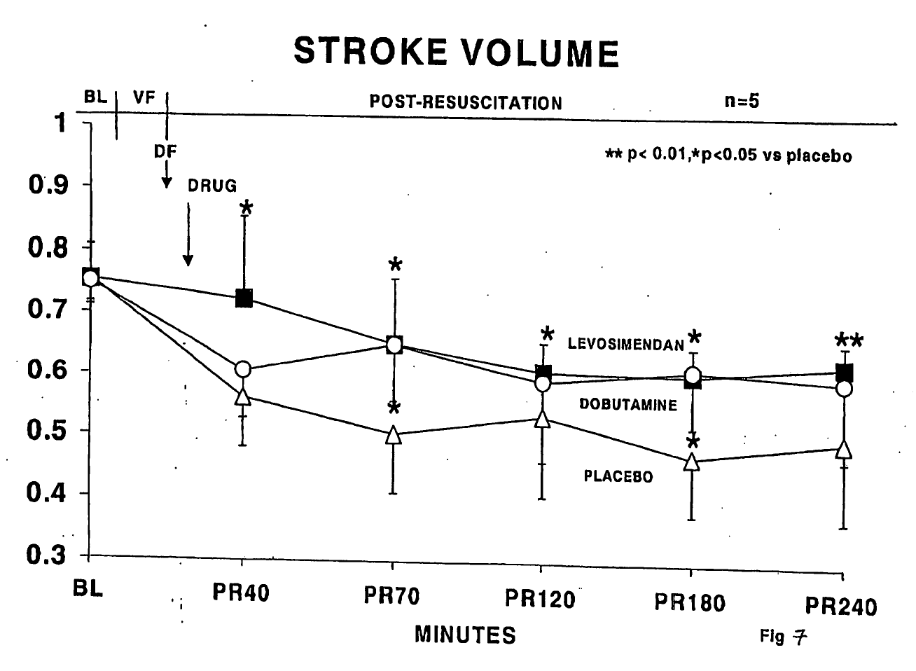

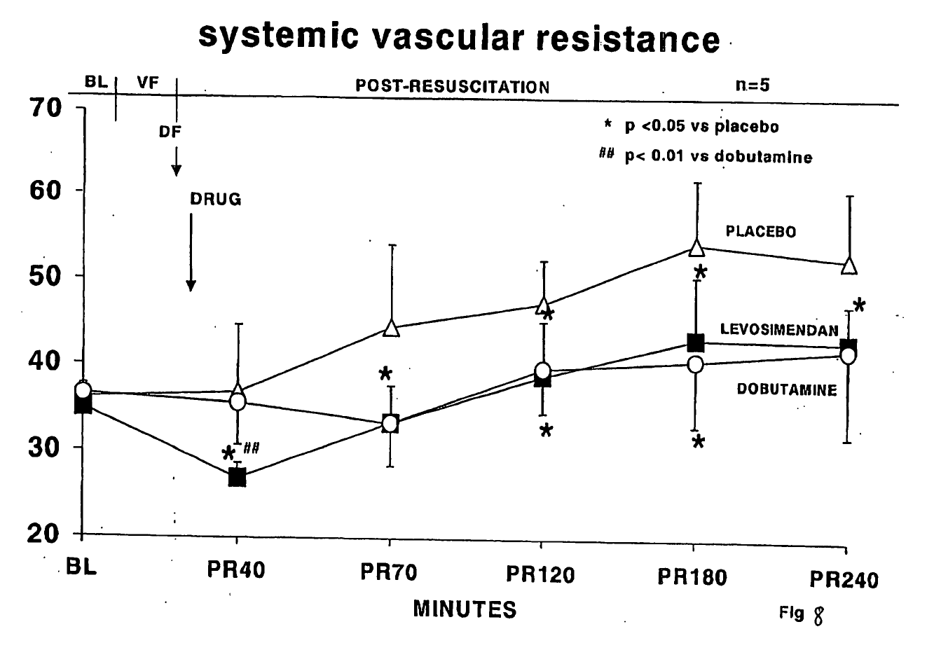

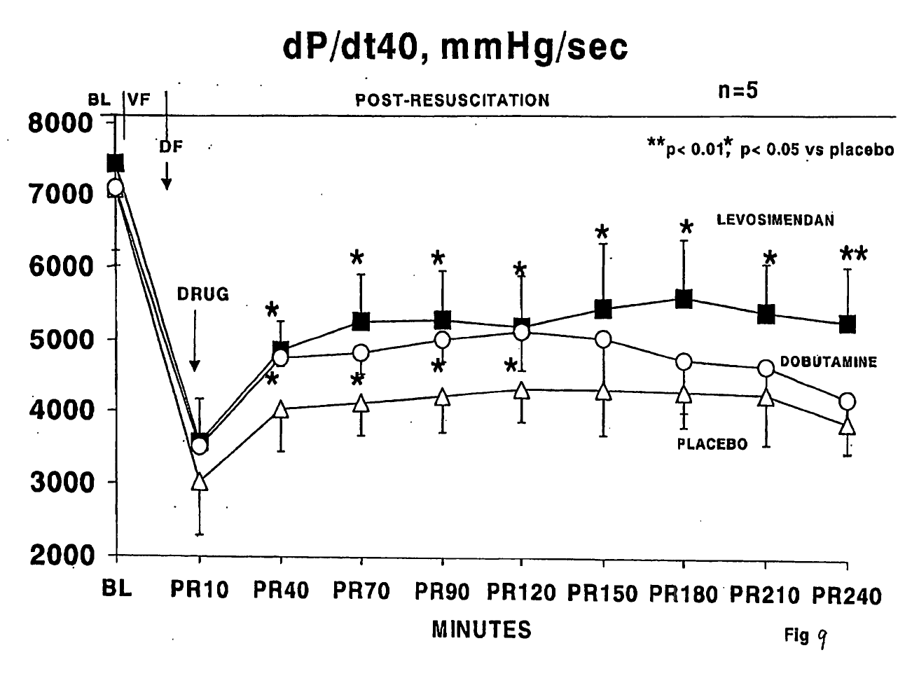

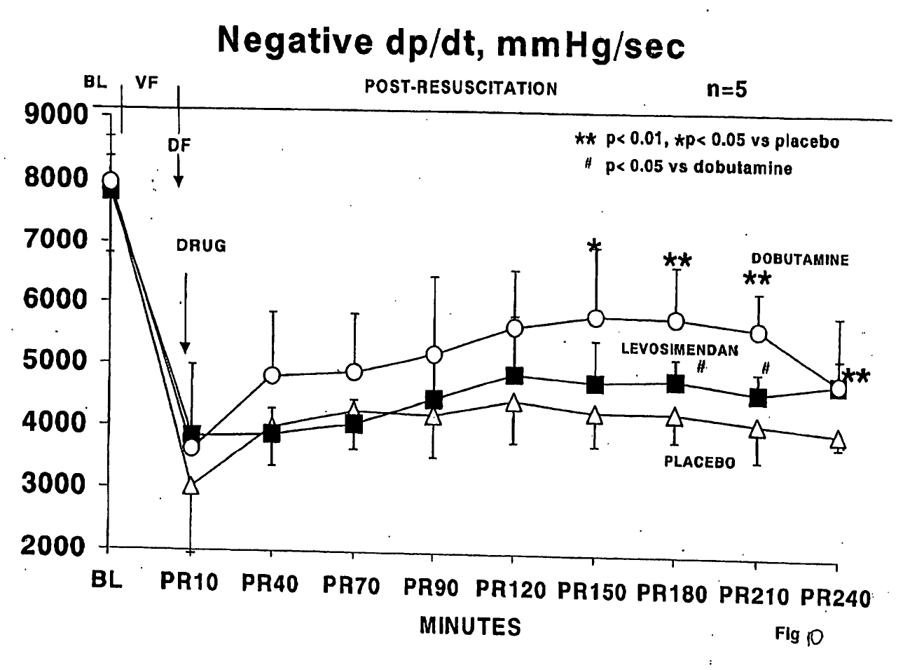

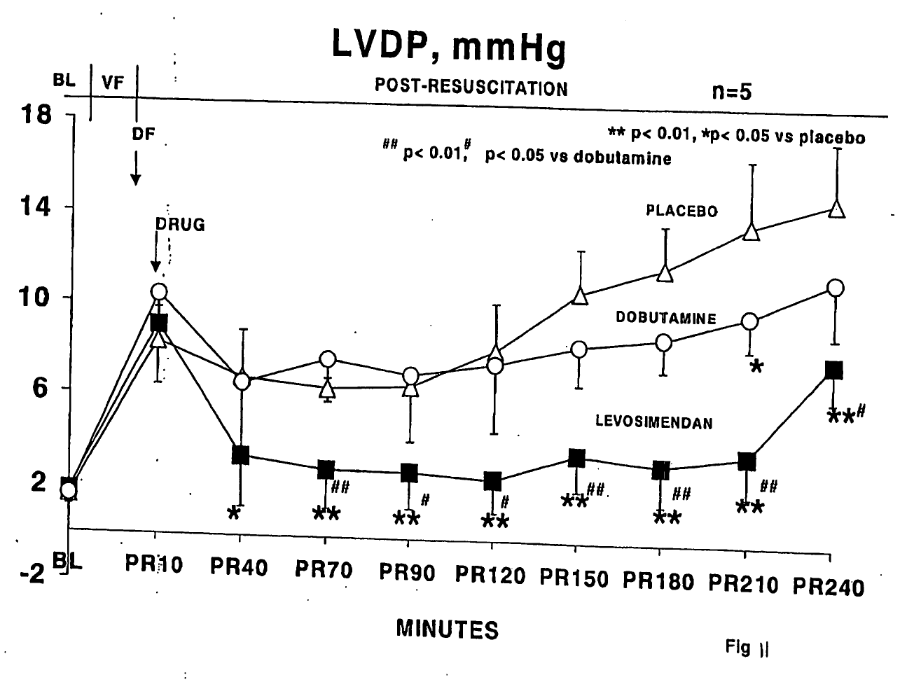

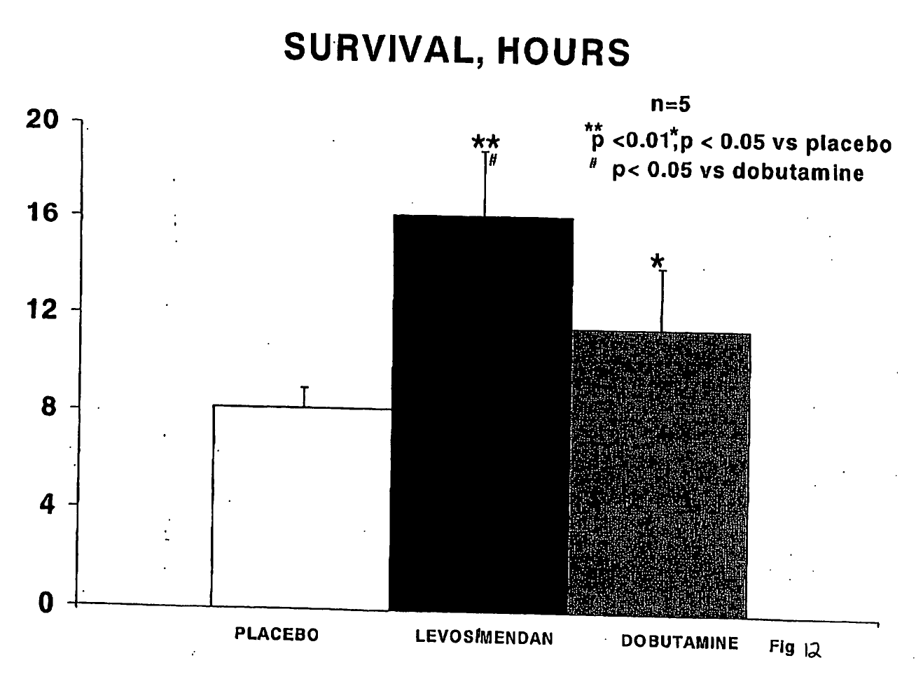

Results: No significant differences were observed in arterial pressures among the three groups (FIG. 4) at 10 minutes after resuscitation (PR10) and after administration of the drugs. Moreover, neither arterial blood gases, arterial blood lactate, nor end-tidal CO₂ were significantly different between the groups, as shown below Tables 1 and 2. Dobutamine produced increases in heart rate of borderline significance (FIG. 5). Both levosimendan and dobutamine yielded comparable increases in cardiac index (FIG. 6), and initially, significantly greater stroke volumes (FIG. 7). Comparable and significant reductions in systemic vascular (arterial) resistance were observed with levosimendan when compared to placebo-treated controls (FIG. 8). Levosimendan yielded consistently greater increases in contractility as reflected in the dP/dt₄₀ (FIG. 9). A more profound lusitropic (relaxation) effect was observed with dobutamine (FIG. 10). However, most striking was the substantially lower and near normal left ventricular diastolic (filling) pressures obtained with levosimendan compared with both control and dobutamine-treated animals (FIG. 11). Finally, there was significantly longer post-resuscitation survival with levosimendan both in comparison with dobutamine and especially in comparison with the placebo (FIG. 12).

TABLE 1
(Blood Gas/Metabolic Parameters)

| | | | |
|---|---|---|---|
| BL | PR40 | PR120 | PR240 |

pHa, units

| | | | | |
|---|---|---|---|---|
| Placebo | 7.51 ± 0.01 | 7.38 ± 0.09 | 7.39 ± 0.03 | 7.42 ± 0.04 |
| Dobutamine | 7.51 ± 0.02 | 7.40 ± 0.05 | 7.39 ± 0.04 | 7.41 ± 0.05 |
| Levosimendan | 7.50 ± 0.01 | 7.40 ± 0.02 | 7.40 ± 0.04 | 7.39 ± 0.03 |

PaCO2, mmHg

| | | | | |
|---|---|---|---|---|
| Placebo | 33.9 ± 2.8 | 27.8 ± 4.6 | 29.5 ± 6.0 | 27.7 ± 5.3 |
| Dobutamine | 36.0 ± 4.6 | 36.6 ± 3.8 | 39.2 ± 4.5 | 37.4 ± 5.9 |
| Levosimendan | 36.9 ± 4.0 | 32.3 ± 6.7 | 33.4 ± 6.2 | 30.7 ± 8.7 |

PaO2, mmHg

| | | | | |
|---|---|---|---|---|
| Placebo | 97.0 ± 8.4 | 405.0 ± 54 | 390.5 ± 58.6 | 363.8 ± |
| | | | | 129.2 |
| Dobutamine | 100.6 ± 13.5 | 342.2 ± 62.7 | 389.9 ± 62.3 | 380.5 ± 40.2 |
| Levosimendan | 105.5 ± 7.3 | 328.2 ± 71.7 | 383.0 ± 43 | 391.6 ± 60.4 |

aLactate, mmol/L

| | | | | |
|---|---|---|---|---|
| Placebo | 0.9 ± 0.3 | 7.4 ± 1.2 | 2.0 ± 0.9 | 1.9 ± 1.2 |
| Dobutamine | 0.9 ± 0.4 | 2.2 ± 0.6 | 1.2 ± 0.4 | 1.1 ± 0.5 |
| Levosimendan | 0.8 ± 0.1 | 3.9 ± 1.8 | 1.6 ± 0.6 | 1.4 ± 0.6 |

TABLE 2
(End Tidal CO₂ [EtCO₂], mmHg)

| | BL | PR10 | PR40 | PR70 | PR120 | PR180 | PR240 |
|---|---|---|---|---|---|---|---|
| Placebo | 37 ± 1 | 29 ± 1 | 35 ± 9 | 33 ± 6 | 34 ± 6 | 32 ± 7 | 29 ± 6 |
| Levosimendan | 38 ± 1 | 33 ± 6 | 30 ± 6 | 35 ± 3 | 34 ± 4 | 32 ± 5 | 30 ± 7 |
| Dobutamine | 38 ± 1 | 33 ± 5 | 35 ± 3 | 35 ± 4 | 38 ± 1 | 36 ± 2 | 35 ± 3 |

### EXAMPLE 2

### Comparison between Dobutamine and Levosimendan in Treating Post-Resuscitation Myocdardial Failure in Rats

Dobutamine is widely used for management of myocardial contractile failure following resuscitation from prolonged cardiac arrest. However, dobutamine has the potential of increasing the severity of ischemic myocardial injury. Levosimendan, an alternative inotrope, has the potential advantage of improving myocardial contractility without increasing the severity of ischemic injury. Accordingly, experiments were understaken to determine whether levosimendan would mitigate postresuscitation myocardial ischemic injury and improve outcomes in comparison with both dobutamine and placebo when administrated after resuscitation from cardiac arrest.

Animal preparation: Fifteen male Sprague-Dawley rats 450 and 550 g were fasted overnight except for free access to water. The animals were anesthetized following intraperitoneal injection of 45 mg kg⁻¹ pentobarbital. Additional intraperitoneal doses of 10 mg kg⁻¹ were administrated at intervals of approximately one hour or as required to maintain anesthesia. No anesthetic agent was administrated during the 30 minute interval prior to inducing cardiac arrest.

The trachea was orally intubated with a 14-gauge cannula mounted on a blunt needle (Abbocath-T; Abbott Hospital Inc., North Chicago, Ill.) with a 145° angled tip by the methods previously described. ²⁰ End-tidal PCO₂ (P_{ET}CO₂) was measured with a side-stream infrared CO₂ analyzer (model 200; Instrumentation Laboratories, Lexington, Mass.) interposed between the tracheal cannula and the ventilator to confirm appropriate minute ventilation. A 23-gauge polyethylene catheter (PE 50, Becton-Dickinson, Sparks, Md.) was advanced into the left ventricle from the surgically exposed right carotid artery for measurement of left ventricular pressure, dP/dt₄₀, and negative dP/dtₘₐₓ. Pressures were measured with a high sensitivity pressure transducer (Model 42584-01; Abbott Critical Care System, North Chicago, Ill.). The optimally damped frequency response of the system was 22 Hz. A 23-gauge polyethylene catheter (PE 50) was advanced through the left external jugular vein, through the superior vena cava into the right ventricle. Guided by pressure monitoring, the catheter was slowly withdrawn into the right atrium. Right atrial pressure was measured with reference to the mid-chest with another high sensitivity pressure transducer (Abbott model 42584-01). This catheter also served as an injection site for the thermal tracer. A 4 F polyethylene catheter (model C-PMS401J; Cook Critical Care, Bloomington, Ind.) was advanced through the right external jugular vein into the right atrium. A precurved guide wire supplied with the catheter was then advanced through the catheter into the right ventricle until an endocardial electrogram was observed. Another 23-gauge polyethylene catheter (PE 50) was advanced through the left femoral artery into the abdominal aorta for measurement of aortic pressure with the same Abbott high sensitivity transducer and also for sampling arterial blood. A thermocouple microprobe, 10 cm in length and 0.5 mm in diameter (9030-12-D-34; Columbus Instruments, Columbus, Ohio) was inserted into the right femoral artery and advanced into the ascending aorta. The thermocouple provided for measurements of blood temperature and thermodilution cardiac output. Another PE 50 catheter was advanced through the left femoral vein, into the inferior vena cava for sampling venous blood and for administrating blood transfusion. An additional PE 50 catheter was advanced into right femoral vein for drug infusion. EKG lead II was continuously recorded.

Experimental Procedure: A total of 15 animals were investigated. The investigators were blinded to the intervention until immediately prior to inducing VF, at which time the principal investigator opened a sealed envelope for assignment to one of three groups: (1) levosimendan (2) dobutamine or (3) saline placebo. This allowed time for preparation of a fresh dilution of the selected drug. VF was induced with a 60-Hz current, which was progressively increased from 2.0 to a maximum of 5.0 mA. Current flow was continued for 3 minutes to prevent spontaneous defibrillation as previously described (Von Planta I, Weil MH. Cardiopulmonary resuscitation in the rat. J Appl Physiol. 1988;65(6):2641-2647). Ventilation was discontinued after onset of VF. Precordial compression was begun with a pneumatically driven mechanical chest compressor after 8 minutes of untreated VF and continued for 6 minutes. These methods have been extensively exercised and have been well-documented (see Von Plata I (supra)) and Sun SJ, Weil MH, Tang W, et al. Combined effects of buffer and adrenergic agent on postresuscitation myocardial function. J Pharm Exp Ther. 1999;291 :773-777). Coincident with the start of precordial compression, the animals were mechanically ventilated. Tidal volume was established at 6.5 ml per kg animal weight, a frequency of 100 min⁻¹, and on a FiO₂ of 1.0. Precordial compression was maintained at a rate of 200 min⁻¹ and synchronized to provide a compression/ventilation ratio of 2:1 with equal compression-relaxation duration. Depth of compression was initially adjusted such as to secure a coronary perfusion pressure (CPP) of approximately 24 mm Hg. This typically yielded a P_{ET}CO₂ of approximately 14 mm Hg (Von Plata I, supra). After 6 minutes of precordial compression, defibrillation was attempted with up to three (3), 2 joule DC electrical shocks. If animals were not resuscitated, precordial compression was resumed for 30 seconds followed by another sequence of electrical shocks. Restoration of spontaneous circulation (ROSC) was defined as the return of supraventricular rhythm with a mean aortic pressure of 60 mm Hg for a minimum of 5 minutes. At 10 minutes after ROSC, one of the three interventions was begun. Doses of levosimendan and dobutamine that were previously shown to be therapeutic (in settings of acute decompensated heart failure), and at the same time did not alter arterial pressure, were selected. Levosimendan was administered in a loading dose of 12 μg kg⁻¹ infused over 10 minutes followed by a 230-minute infusion of 0.3 μg kg⁻¹ min⁻¹. Dobutamine was infused into the right atrium in an amount of 3 μg kg⁻¹ min⁻¹ lover an interval of 240 minutes. The saline placebo was infused in total volumes of 5 ml over the 240-minute interval, in an amount that was equal to that of both dobutamine and levosimendan. A syringe pump (Model 940, Harvard Apparatus, Southnatick. Mass.) was utilized. Mechanical ventilation with oxygen and hemodynamic measurements were continued for a total of 4 hours after successful resuscitation. Animals were allowed to recover from anesthesia after 4 hours whereupon all catheters, together with the endotracheal tube were then removed and the animals were allowed to breathe room air. Survival was observed over the ensuring 72 hours. After 72 hours, animals were euthanized and autopsy was routinely performed. Organs were inspected for gross abnormalities, including evidence of traumatic injuries consequent to cannulation, airway management, or precordial compression.

Measurements: PO₂, PCO₂, pH, SO₂ and lactate, calcium and blood glucose were measured on 0.5 mL samples of arterial and venous blood by techniques previously described.^{4,21} A 1.0-mL bolus of arterial blood from an anesthetized donor rat of the same colony was transfused into the inferior vena cava in an amount equivalent to the two 0.5-ml aliquots withdrawn from the aorta and the femoral vein for the laboratory measurements. Measurements were obtained at baseline, at 30, 120 and 240 minutes after successful resuscitation. Aortic, left ventricular, and right atrial pressures, EKG, and P_{ET}CO₂ were continuously recorded on a PC-based data acquisition system supported by CODAS software (DATAQ Inc., Akron, Ohio). CPP was calculated as the difference between decompression diastolic aortic and time-coincident right atrial pressure measured at the end of each minute of precordial compression.

The rate of left ventricular pressure increase (dP/dt₄₀) was measured by differentiation at a left ventricular pressure at 40 mm Hg and served as a quantitative estimate of isovolumic contractility. The rate of maximal left ventricular pressure decline, the −dP/dt, was also measured together with left ventricular diastolic pressures as an estimate of myocardial lusitropy.^{20,21} Cardiac output was measured by the thermodilution method with the aid of a cardiac output computer (CO-100;

Institute of Critical Care Medicine, Palm Springs, Calif.) at baseline and at 30, 60, 180 and 240 minutes after successful resuscitation. In each instance duplicate measurements differed by no more than 5%.

Statistical Analyses: For measurements between groups, ANOVA and Scheffe's multicomparision techniques were employed. The outcome differences were analyzed with Fisher's exact test. Measurements are reported as mean ±SD. A value of P<0.05 was considered significant.

Results: No significant differences in baseline values of heart rate, arterial pressure, left ventricular diastolic pressure, dP/dt₄₀, negative dP/dt, cardiac index, and ETCO₂ were observed (Table 3). There were also no significant differences in arterial and venous blood gas, lactate, calcium or blood glucose. Each animal was successfully resuscitated after 14 minutes of cardiac arrest, including 8 minutes of untreated VF followed by 6 minutes of precordial compression and mechanical ventilation.

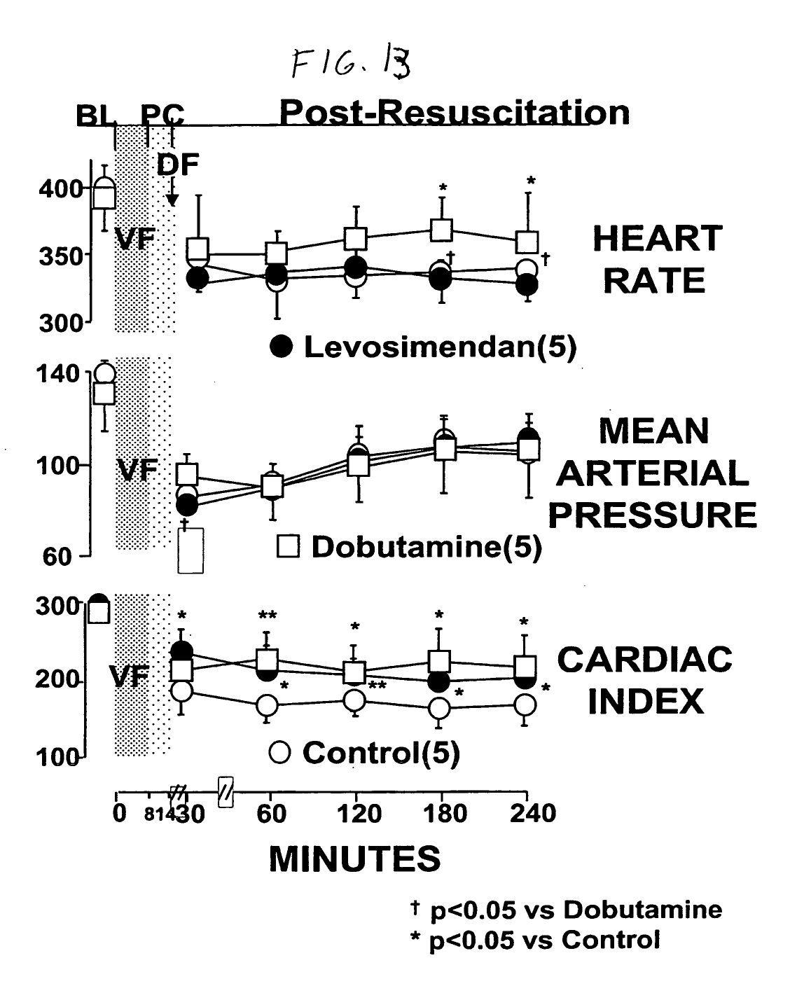

A moderate increase in heart rate was observed after administration of dobutamine, in accord with the anticipated response to the doses administered. However, there were no significant differences in mean arterial pressure (MAP) between the three groups. As anticipated, both dobutamine and levosimendan but not placebo produced significant increases in cardiac index. (FIG. 13)

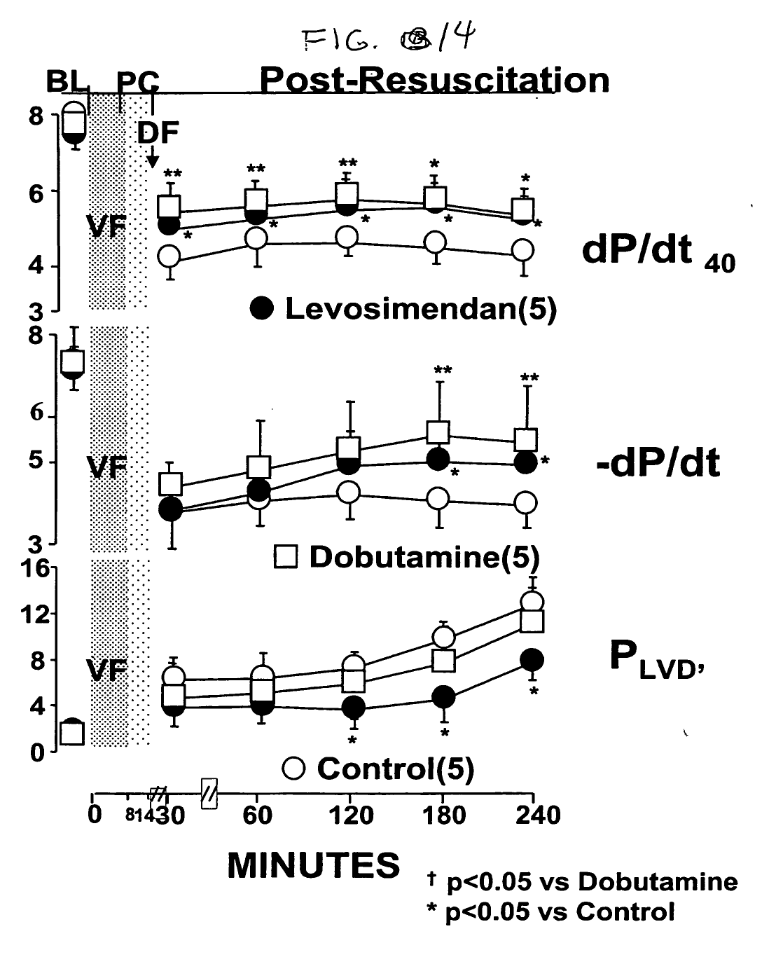

Both dobutamine and levosimendan improved contractile and lusitropic functions as shown in FIG. 14. Significantly greater dP/dt₄₀ and −dP/dt were demonstrated in comparison with saline placebo. Levosimendan produced significantly lesser increases in the left ventricular diastolic (filling) pressures. Significantly greater arterial PCO₂ and ETCO₂ were noted in dobutamine-treated animals between the second and fourth hour that followed ROSC. In addition, a consistently lower arterial oxygen saturation after dobutamine was observed, although the differences were not statistically significant (Table 4). However, no consistent differences in arterial and mixed venous pH, PO₂, lactate, glucose or calcium were identified.

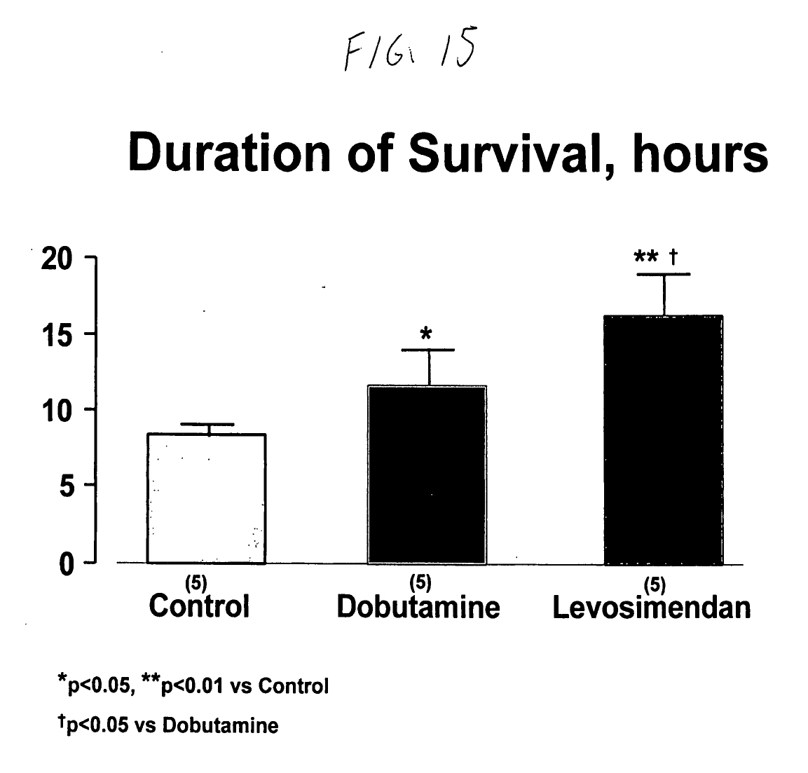

The most important finding was an increase in the duration of survival, which was maximal with levosimendan, intermediate with dobutamine, and least with saline placebo. The differences between levosimendan and both dobutamine and saline placebo were significant as shown in FIG. 15. Autopsy recorded no gross injuries to the thoracic or abdominal viscera.

This experimental comparison demonstrates that the administration of levosimendan following resuscitation from cardiac arrest improves postresuscitation myocardial function comparable to that produced by dobutamine. However, there is greater survival benefit with levosimendan in association with lesser increases in heart rate and more favorable left ventricular filling pressures.

### EXAMPLE 3

### Comparison between Dobutamine and Levosimendan for Treatment of Post Resuscitation Myocdardial Failure in Pigs

Experimental Preparation: The experiments were performed in the porcine model of cardiac arrest and cardiac resuscitation which has been extensively exercised. (21,22). Briefly, 15 male domestic pigs weighing between 35 and 40 kg were fasted overnight except for free access to water. Anesthesia was initiated by intramuscular injection of ketamine (20 mg kg⁻¹) and was completed by ear vein injection of sodium pentobarbital (30 mg kg⁻¹). Additional 8 mg kg⁻¹ doses of sodium pentobarbital were injected to maintain anesthesia at intervals of one hour. A cuffed endotracheal tube was advanced into the trachea. Animals were mechanically ventilated with a volume of 15 mL kg⁻¹, peak airway flow of 40 L min⁻¹, and FiO₂ of 0.2 with the aid of a volume-controlled ventilator (Model MA-1, Puritan-Bennett, Carlsbad, Calif.). End-tidal PCO₂ (P_{ET}CO₂) was monitored with an infrared analyzer (Model 01R-7101A, Nihon Kohden Corp, Tokyo, Japan). Respiratory frequency was adjusted to maintain P_{ET}CO₂ between 35 and 40 mmHg. Blood temperature was maintained at 37±0.5° C. with the aid of infrared heating lamps, as required.

For the measurement of left ventricular functions, a 5.5/7.5 MHz biplane with pulse-wave Doppler transesophageal echocardiographic tranducer with 4-way flexure (Model 21363A, Hewlett-Packard Co. Medical Products Group, Andover, Mass.) was advanced from the incisor teeth into the esophagus for a distance of approximately 40 cm. For the measurement of aortic pressure, a fluid filled catheter was advanced from the surgically exposed left femoral artery into the thoracic aorta. For measurements of right atrial and pulmonary arterial pressures, blood temperature and cardiac output, a 7-French, pentalumen, thermodilution-tipped catheter was advanced from the surgically exposed left femoral vein and flow-directed into the pulmonary artery. For inducing VF, a 5-French pacing catheter (EP Techologies, Inc., Mountain View, Calif.) was advanced from the surgically exposed right cephalic vein into the right ventricle. Through the surgically exposed left cephalic vein, a 7-French angiographic cathether (5470, USCI C.R. Bard, Murray Hill, N.J.) was advanced with the aid of fluoroscopy through the superior vena cava into the right atrium and the coronary sinus. This catheter was then looped laterally and advanced inferiorly for a distance of 5 cm into the great cardiac vein for sampling of coronary venous blood. The electrocardiographic (EKG) lead II was continuously recorded.

Experimental procedure: A total 15 animals were investigated. Fifteen minutes prior to induction of VF, the animals were randomized by the sealed envelope method. Cardiac arrest was induced with 1 to 2 mA AC delivered to the endocardium of the right ventricle. Mechanical ventilation was discontinued after onset of VF. At the end of a 7-minute interval of untreated VF, precordial compression (PC) was started with a pneumatic piston-driven chest compressor (Thumper, Model 1000, Michigan Instruments, Grand Rapids, Mich.). Coincident with the start of PC, the animal was mechanically ventilated with a tidal volume of 15 mg kg⁻¹ and FiO₂ of 1.0. PC was programmed to provide 100 compressions per minute and synchronized to provide a compression/ventilation ratio of 5:1 with equal compression-relaxation intervals, i.e. a 50% duty cycle. The compression force was adjusted to decrease the anterior-posterior diameter of the chest by 25%. After 5 minutes of PC, defibrillation was attempted with a 150 J biphasic waveform shock delivered between the right infraclavicular area and the cardiac apex. If an organized cardiac rhythm with mean aortic pressure of more than 60 mm Hg persisted for an interval of 5 minutes or more, the animal was regarded as successfully resuscitated. At 10 minutes after restoration of spontaneous circulation (ROSC), one of the three interventions was begun. Doses of levosimendan and dobutamine were administered in accord with earlier trials (23-25) and after it was confirmed that these doses did not alter mean arterial pressure in normal anesthetized pigs under physiological conditions. Levosimendan diluted in physiological salt solution was administered in a loading dose of 20 μg kg⁻¹, infused over 10 minutes, followed by infusion of 0.4 μg kg⁻¹ min⁻¹ also in physiologic salt solution for a total duration of 230 minutes. Dobutamine, diluted in physiological salt solution, was infused into right atrium in an amount of 5 μg kg⁻¹ min⁻¹ for a total interval of 240 minutes. An equivalent volume of physiological salt solution without the drug was infused over 10 minutes after ROSC followed by a 230-minute continuous infusion in volume equivalent to that of levosimendan and dobutamine. Mechanical ventilation with 100% oxygen together with hemodynamic measurements were continued for a total 4 hours after resuscitation. Thereafter, animals were allowed to recover from anesthesia and all catheters, including the endotracheal tube, were removed after 4 hours. At the end of the 72-hour observation interval, animals were euthanized and an autopsy was routinely performed. At autopsy, organs were inspected for gross abnormalities, including evidence of traumatic injuries consequent to cannulation, airway management, or precordial compression.

Measurements: Hemodynamic data, including aortic, right atrial, and mean pulmonary artery pressures, coronary perfusion pressure, end-tidal PCO₂, and lead 2 of the EKG were continuously monitored in real time and recorded on a PC-based data acquisition system, supported by CODAS hardware/software (DATAQ Inc., Akron, OH) as previously described (21,22).

Echocardiographic measurements were obtained with the aid of a transesophageal echocardiographic transducer with 4-way flexure. Left ventricular end-systolic and end-diastolic volumes were calculated from the long axis view by the method of discs (Acoustic Quantification Technology, Hewlett-Packard, Andover, Mass.). From these, ejection fraction and the fractional of area change were computed. These measurements served as quantitators of myocardial contractile function. Measurements are reported for baseline, 30, 60, 120, 180, and 240 minutes after successful resuscitation.

Arterial blood gases were measured on 200 μL aliquots of blood with a stat profile analyzer (ULTRA C, Nova Biomedical Corporation, Waltham, Mass.) adapted for porcine blood. Neurological alertness was scored on a scale of 100 (fully alert and active) to 0 (non-reactive with apnea) as previously described (22). In addition to alertness and activity, the score includes posture, water and food intake, and objective signs of self-care at 24 hours, 48 hours and 72 hours after resuscitation from cardiac arrest.

Statistical Analysis: For measurements between groups, ANOVA and Scheffe's multicomparision techniques were employed. The outcome differences were analyzed with Fisher's exact test. Measurements are reported as mean±SD. Ap value of <0.05 was considered significant.

Results: No significant differences in baseline values of heart rate (HR), mean arterial pressure (MAP), right atrial pressure (RAP), mean pulmonary arterial pressure ejection (MPAP), ejection fraction (EF), fraction of area change (FAC), cardiac output (CO), and end-tidal PCO₂ (P_{ET}CO₂) were observed. There were also no significant differences in the values of baseline blood gas measurements. Each animal was successfully resuscitated after 7 minutes of untreated VF, after 5 minutes of precordial compression and mechanical ventilation, representing a total of 12 minutes of cardiac arrest.

Heart rate did not differ among the three groups. As anticipated, there were no differences in arterial pressure between levosimendan and dobutamine, but significantly lower arterial pressure was observed with saline placebo at 60 minutes after resuscitation. Significantly lower mean pulmonary artery and right atrial (filling) pressures were observed in the 4-hour interval after treatment with levosimendan. (Table 5).

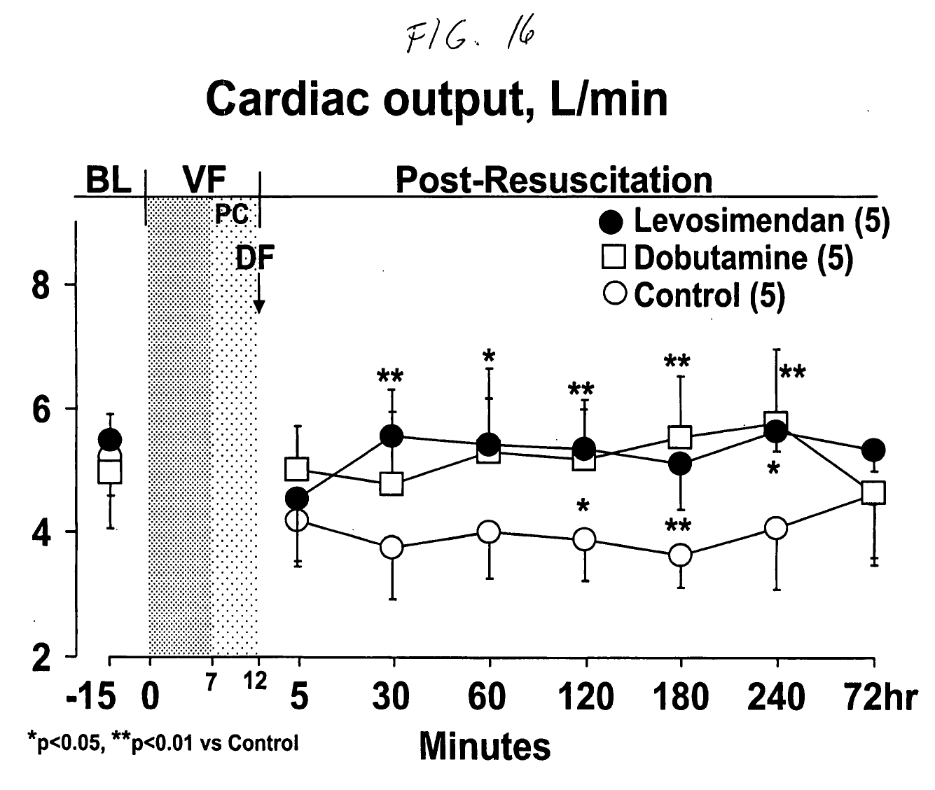

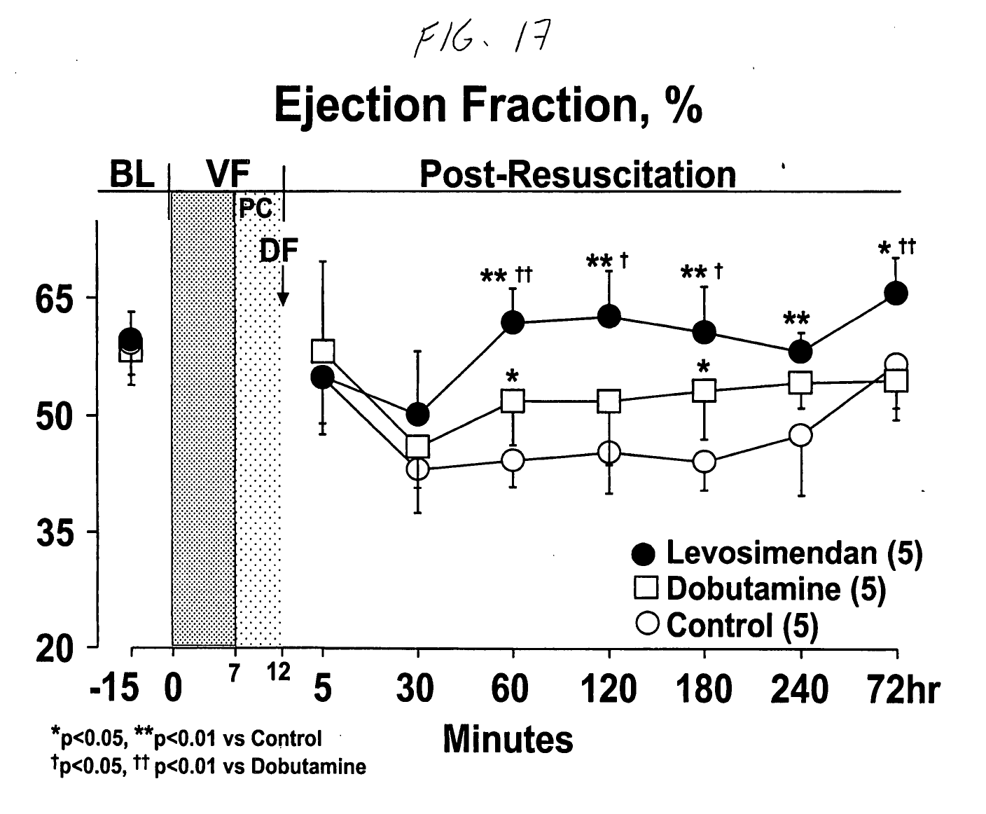

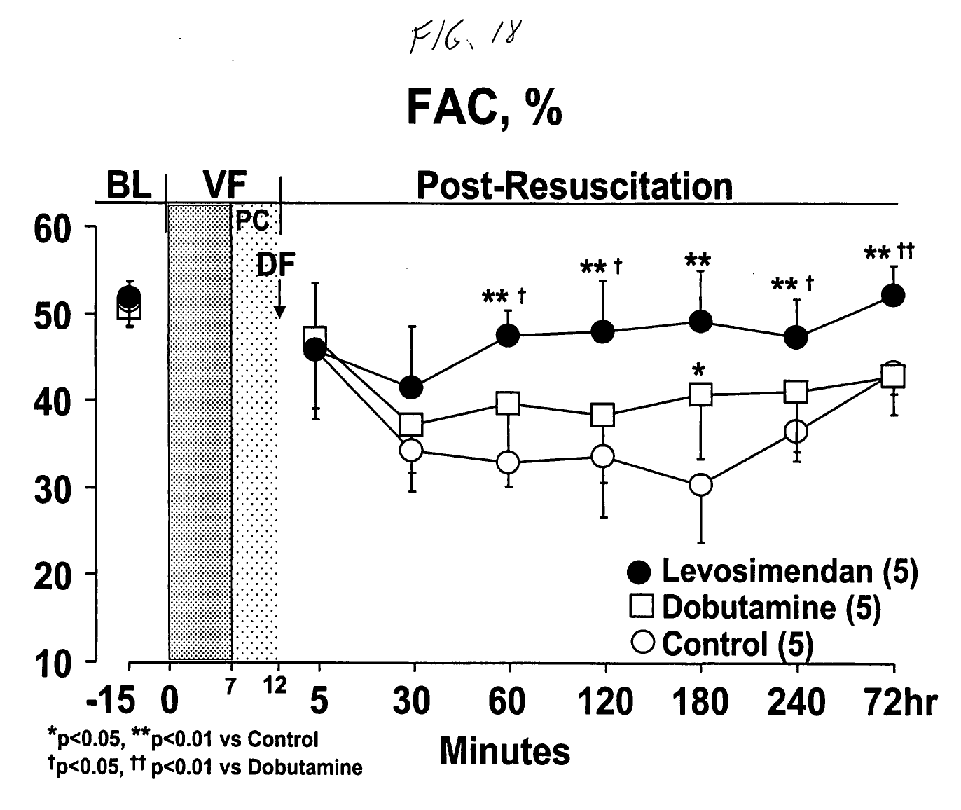

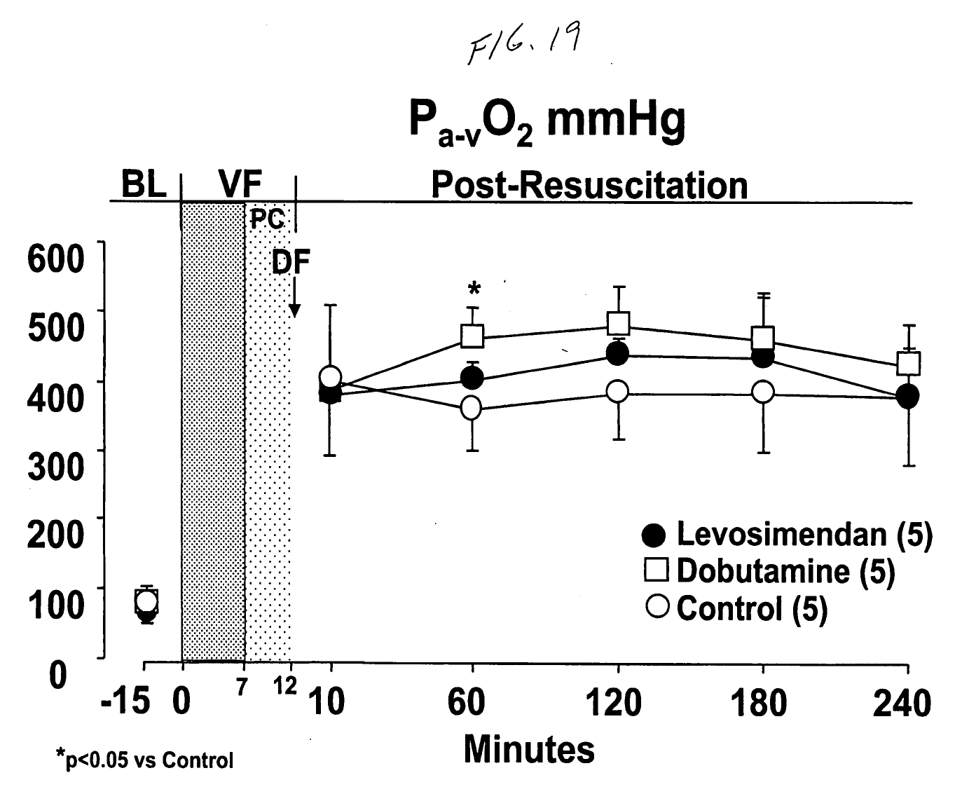

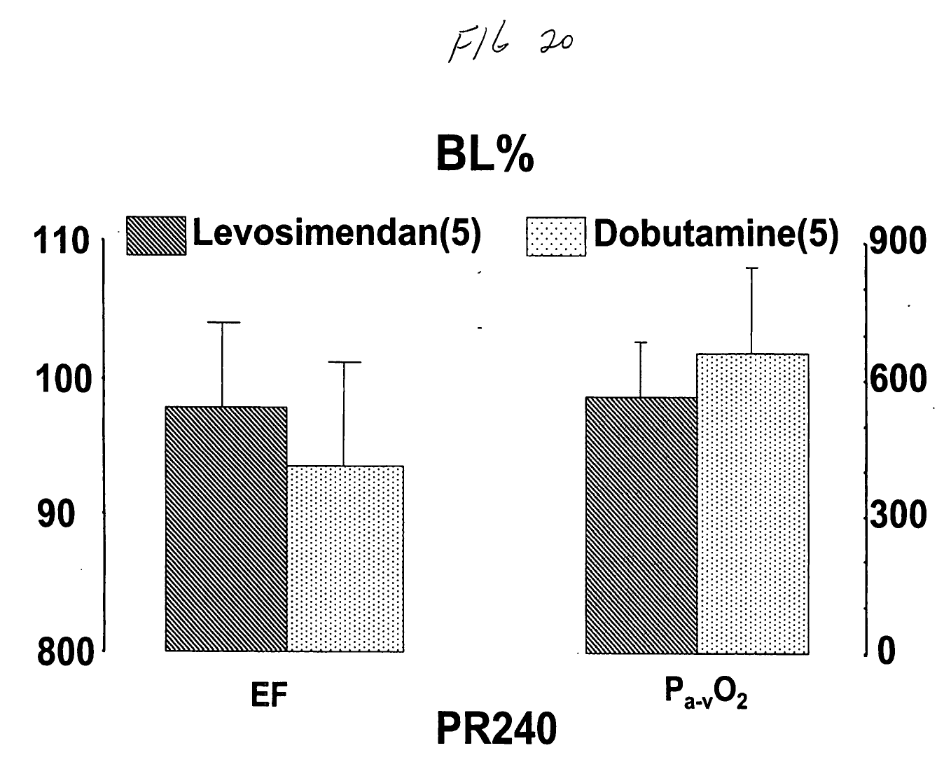

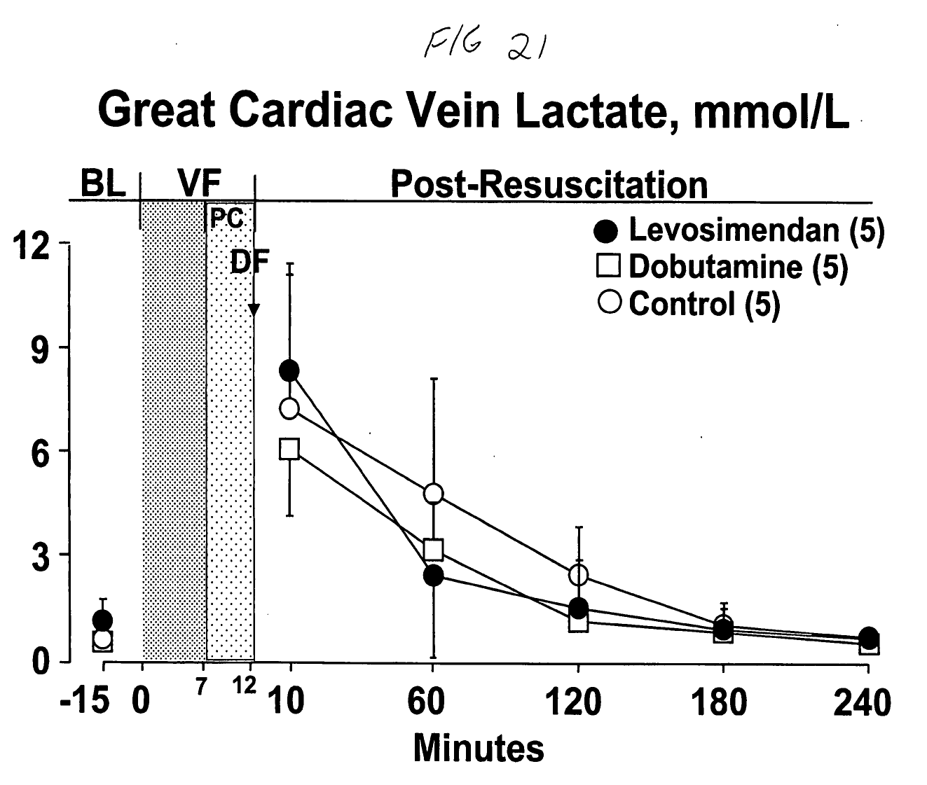

Both levosimendan and dobutamine improved contractile function in the doses administered. Significantly greater cardiac output was demonstrated for both inotropes in comparison to saline placebo as shown in FIG. 16. However, levosimendan resulted in significantly greater EF and FAC which persisted at 72 hours (FIGS. 17 and 18) with numerically smaller coronary arterial-venous oxygen differences (FIG. 19) in comparison to dobutamine. Accordingly, increases in contractility in terms of ejection fraction were observed without increases in oxygen extraction (FIG. 20). No differences in coronary venous lactate were noted (FIG. 21). The neurological alertness scores were significantly better with levosimendan at 24 hours (Table 6).

### EXAMPLE 4

### Effect of Administration of Levosimendan During Cardiopulmonary Resuscitation

Animal Preparation: Ten male Sprague-Dawley rats weighing 450-580 g were fasted overnight except for free access to water. The animals were anesthetized by intraperitoneal injection of pentobarbital (45 mg/kg). Additional doses of 10 mg kg⁻¹ were administrated at intervals of approximately one hour or as required to maintain anesthesia. No anesthetic agents were initially administrated 30 min prior to inducing cardiac arrest. The trachea was orally intubated with a 14 gauge cannula mounted on a blunt needle with a 145° angled tip by the methods of Stark (Stark R A, Nahrwold M L, Cohen P J. Blind oral tracheal intubation of rats. J Appl Physiol: Resp Environ Exercise Physiol 1981 ;51(5): 1355-1356). A polyethylene catheter (PE 50, Becton-Dickinson) was advanced into the left ventricle from the surgically exposed right carotid artery for measurement of left ventricular pressure, including both dP/dt₄₀ and negative dP/dt. A polyethylene catheter (PE 50, Becton-Dickinson) was advanced through the left external jugular vein and the superior vena cava into the right atrium. Right atrial pressure was measured with a high-sensitivity pressure transducer (model 42584-01; Abbott Critical Care System, North Chicago, Ill.). A thermocouple microprobe, 10 cm in length and 0.5 mm in diameter (9030-12-D-34; Columbus Instrument, Columbus, Ohio), was inserted into the right femoral artery and advanced into the descending thoracic aorta. Blood temperature was measured with this sensor. For cardiac output measurements, 0.2 mL of isotonic saline, with temperature ranging between 8 and 12° C., was injected into the right atrium through the catheter advanced from the left jugular vein. Duplicate thermodilution curves were obtained and recorded with the aid of a cardiac output computer (CO-100; Institute of Critical Care Medicine, Palm Springs, Calif.). A PE 50 catheter was advanced through the left femoral artery into the thoracic aorta for sampling arterial blood for analysis of blood gases and for the measurement of aortic pressure with a high-sensitivity pressure transducer (model 42584-01; Abbott Critical Care System). Systolic, diastolic, and the interpreted mean arterial pressure were continuously recorded. Another PE 50 catheter was advanced through the left femoral vein into the inferior vena cava for blood sampling to provide to analysis of venous blood gases. A 1.2 mL bolus of arterial blood from a donor rat of the same colony was transfused into the inferior vena cava immediately after withdrawal of a total of 0.6 mL aliquots of blood, each from the aorta and inferior vena cava. A 4F polyethylene catheter (model C-PMS-401J; Cook Critical Care, Bloomington, Ind.) was next advanced through the right external jugular vein into the right atrium for inducing VF. A precurved guide wire, supplied with the catheter, was then advanced through the catheter into the right ventricle until an endocardial electrogram was observed. A 60 Hz AC, to a maximum of 3.5 mA, was delivered to the right ventricular endocardium until VF was induced. Current flow was then reduced to one half and continued for 3 min such as to prevent spontaneous defibrillation. VF was untreated for six min. Mechanical ventilation was stopped after onset of VF. Precordial compressions were performed with a pneumatically driven mechanical chest compressor. These procedures were previously described in greater detail (Von Planta I, supra) and are well-known to those of ordinary skill in the art (see e.g. Tang W, Weil M H, Sun S, Noc M, Yang L, Gazmuri R. Epinephrine increases the severity of postresuscitation myocardial dysfunction. Circulation 1995; 92: 3089-3093 and Sun S, Weil MH, Tang W, Povoas H, Mason E. Combined effect of buffer and adrenergic agent on postresuscitation myocardial function. Pharmacology 1999; 291 (2): 773-777).

Coincident with start of precordial compression, the animals were mechanically ventilated. Tidal volume was established at 0.65 mL/100 g animal body weight and at a frequency of 100/min, and with a FiO₂ of 1.0. Precordial compressions were maintained at a rate of 200/min and synchronized to provide a compression/ventilation ratio of 2:1 with equal compression-relaxation duration. Depth of compressions was initially adjusted to secure a coronary perfusion pressure (CPP) of 23±1 mm Hg. This typically yielded an end-tidal PCO₂ of 14±3 mm Hg (Von Planta I, supra). Resuscitation was attempted with up to 3 two-joule biphasic shocks. Restoration of spontaneous circulation (ROSC) was defined as the return of supraventricular rhythm with a mean aortic pressure of 60 mm Hg for a minimum of 5 min. Levosimendan, supplied by Orion Corp, Espoo, Finland, in a dilution of 2.5 mg/mL, was injected into the right atrium after two minutes of untreated VF in a bolus dose of 20 μg/kg. Mechanical ventilation with oxygen was continued for 4 hours after resuscitation. Animals were then allowed to recover from anesthesia and all catheters, including the endotracheal tube, were removed. Electrocardiographic (EKG) lead II was continuously recorded. After the animals had been returned to their cages, the post-resuscitation activity status of the animal was recorded at 4-hour intervals for a total of 48 hours. Animals were euthanized by intraperitoneal injection of pentobarbital (150 mg/kg) and autopsy was routinely performed to exclude injuries to the bony thorax and the thoracic and abdominal viscera during the CPR intervention.

Statistical Analyses. For measurements between groups, ANOVA and Scheffe's multicomparision techniques were employed. Comparisons between time-based measurements within each group were performed with ANOVA repeated measurement. Categorical variables were analyzed with Fisher exact test. Measurements are reported as mean±SD. Values of p<0.05 were considered significant.

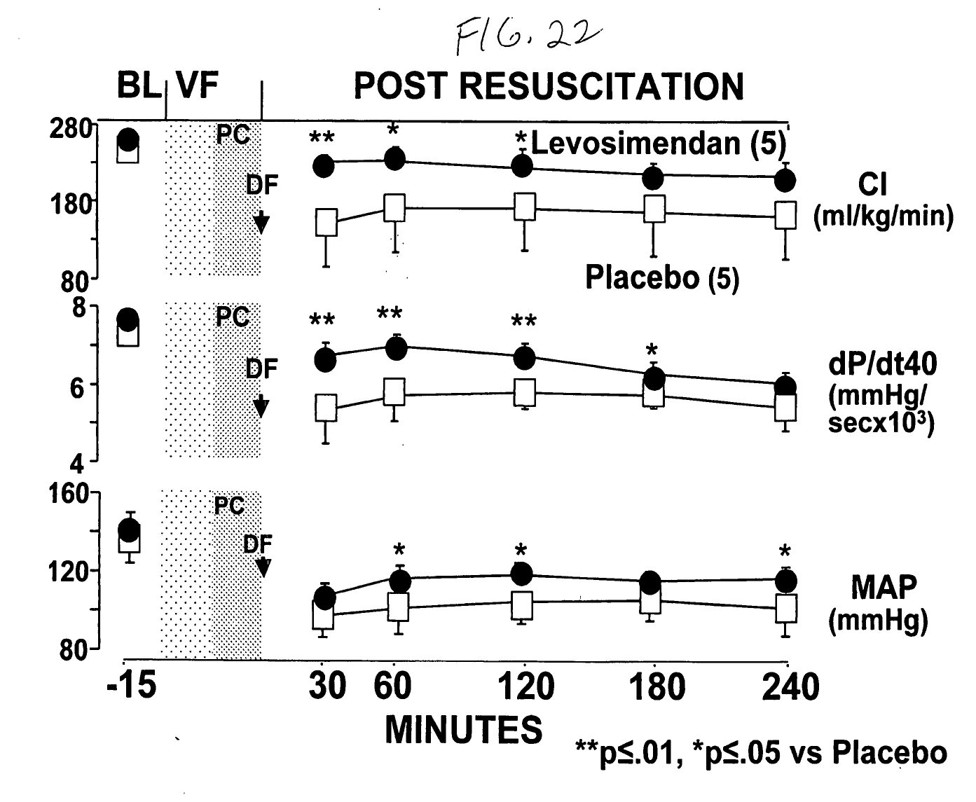

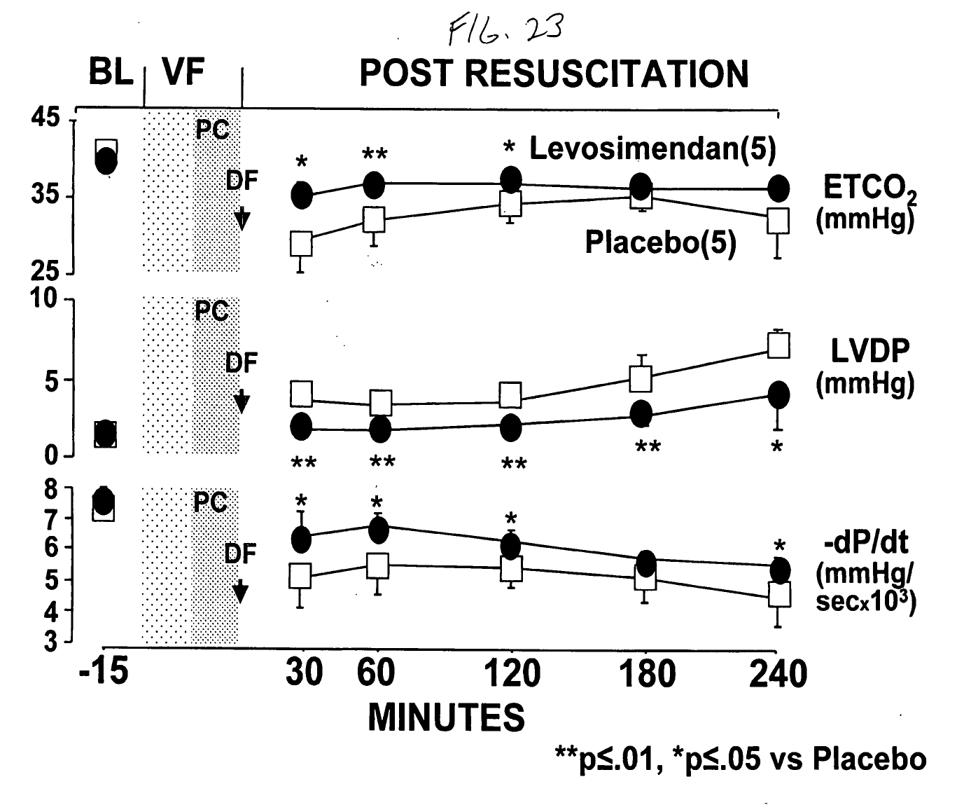

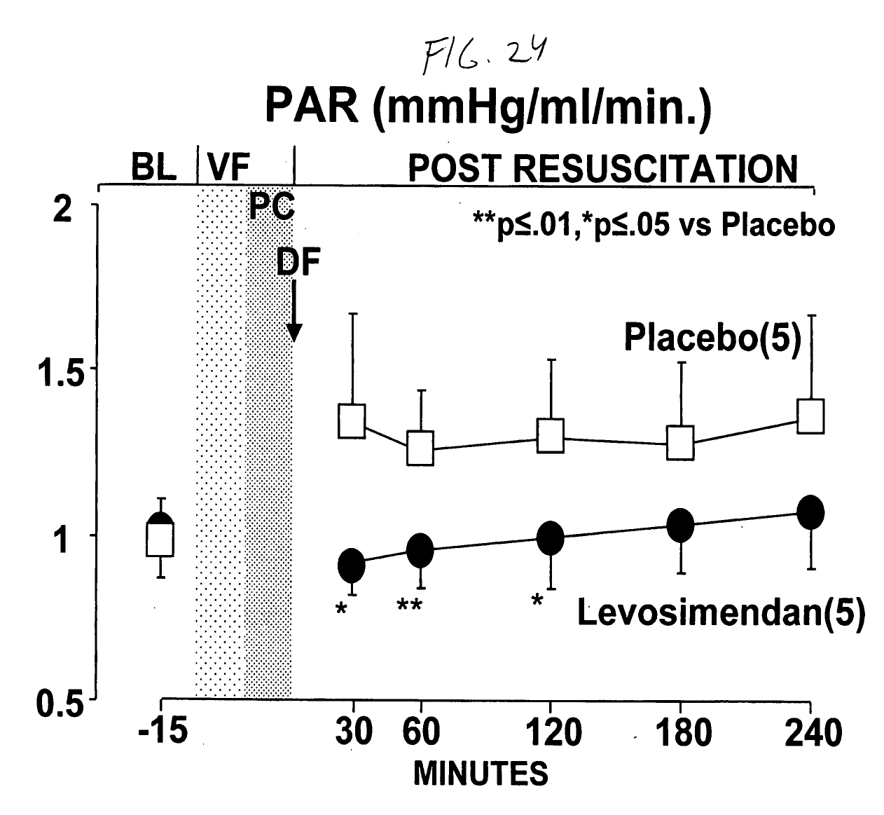

Results: Baseline hemodynamic and blood analysis did not differ significantly among levosimendan and placebo-treated animals. Coincident with the onset of VF, the mean aortic pressure (MAP) decreased from 133±6 to 11±2 mm Hg and the MAP increased from 1±1 to 9±2 mm Hg in confirmation of earlier reports (Tang W, Weil M H, Sun S, Pernat A, Mason E. KATP channel activation reduces the severity of post-resuscitation myocardial dysfunction. Am J Physiol 2000; 279: H1609-H1615). Except for occasional increases induced by agonal gasps, CPP remained between 1 and 3 mm Hg during the 6 min of untreated cardiac arrest. Precordial compression increased CPP to an average of 23±1 mm Hg. No differences in CPP between animals subsequently assigned to levosimendan treatment and to placebo controls were observed either prior to/or after administration of levosimendan. Each animal was successfully defibrillated. However, levosimendan-treated animals required a significantly shorter interval of CPR prior to successful resuscitation (Table 7). The cumulative number of electrical shocks required for successful defibrillation was significantly less after levosimendan than in the 5 placebo-treated animals. Significantly greater cardiac index, dP/dt₄₀ and MAP were documented over the 4-hour interval following resuscitation in the levosimendan-treated animals (FIG. 22). Negative dP/dt as an indicator of left ventricular compliance was increased together with ETCO₂ (FIG. 23). Improved left ventricular function was also reflected in a reduction in left ventricular diastolic pressure (FIG. 23) together with lesser ST segment elevation following levosimendan (Table 7). The peripheral arterial resistance (PAR) was significantly decreased after levosimendan (FIG. 24). The duration of post-resuscitation survival was significantly increased in levosimendan-treated animals (Table 8).

The lesser post-resuscitation ST segment elevations provide additional evidence of the capability of levosimendan to minimize ischemic injury and therefore residual ischemia following successful resuscitation. Since levosimendan reduces peripheral resistance, the consequent reduction in left ventricular after-load would also explain improved systolic function with augmented cardiac index and increases in arterial pressure, even though there are vasodilator or concurrent decreases in peripheral arterial resistance. Each of these measurements summates to improved outcome when levosimendan is administered during cardiac arrest.

### Effect of Levosimendan on Post-Resuscitation Myocardial Function after Beta-adrenergic Blockage

Animal preparation: Male domestic pigs weighing 35 to 40 kg were fasted overnight except for free access to water. Anesthesia was initiated by intramuscular injection of ketamine (20 mg/kg) and completed by ear vein injection of sodium pentobarbital (30 mg/kg). Additional doses of sodium pentobarbital (8 mg/kg) were injected to maintain anesthesia at intervals of one hour. A cuffed endotracheal tube was advanced into the trachea. Animals were mechanically ventilated with a volume-controlled ventilator (Model MA-1, Puritan-Bennett, Carlsbad, Calif.) with a tidal volume of 15 mL/kg, peak flow of 40 L/min, and FiO₂ of 0.21. End-tidal PCO₂ (ETCO₂) was monitored with an infrared analyzer (Model 01R-7101A, Nihon Kohden Corp, Tokyo, Japan). Respiratory frequency was adjusted to maintain P_{ET}CO₂ between 35 and 40 mm Hg.

For the measurement of left ventricular functions, a 5.5/7.5 Hz biplane with Doppler transesophageal echocardiographic transducer with 4-way flexure (Model 21363A, Hewlett-Packard Co., Medical Products Group, Andover, Mass.) was advanced from the incisor teeth into the esophagus for a distance of approximately 35 cm. For the measurement of aortic pressure, a fluid-filled catheter was advanced from the left femoral artery into the thoracic aorta. For the measurements of right atrial, pulmonary arterial pressures and blood temperature, a 7-French, pentalumen, thermodilution-tipped catheter was advanced from the left femoral vein and flow-directed into the pulmonary artery. A 7-French catheter was advanced from the left cephalic vein into the great cardiac vein for the measurement of great cardiac vein blood gases and lactate. For inducing VF, a 5-French pacing catheter (EP Technologies, Inc., Mountain View, Calif.) was advanced from the right cephalic vein into the right ventricle.

Experimental Procedure: Fifteen min prior to VF, the animals were randomized by the sealed envelope method. The investigators were blinded to the randomization. Cardiac arrest was induced with 1 to 2 mA AC delivered to the endocardium of the right ventricle. Mechanical ventilation was discontinued after onset of VF. At the end of the 7-min interval of untreated VF, precordial compression was started with a pneumatic piston-driven chest compressor (Thumper, Model 1000, Michigan Instruments, Grand Rapids, Mich.). Coincident with the start of precordial compression, the animal was mechanically ventilated with a tidal volume of 15 mL/kg and FiO₂ of 1.0. Precordial compression was programmed to provide 100 compressions/min and synchronized to provide a compression/ventilation ratio of 5:1 with equal compression-relaxation intervals, i.e. a 50% duty cycle. The compression force was adjusted to decrease the anterior-posterior diameter of the chest by 25%. After 5 min of precordial compression, defibrillation was attempted with a 150 J biphasic waveform shock delivered between the right infraclavicular area and the cardiac apex. If an organized cardiac rhythm with mean aortic pressure of more than 60 mm Hg persisted for an interval of 5 min or more, the animal was regarded as successfully resuscitated. All animals had restoration of spontaneous circulation (ROSC) after electrical defibrillation, and were then randomized to three treatment groups: (1) propranolol (0.1 mg/kg bolus at 6 min of VF); (2) propranolol plus levosimendan (at 10 min after post resuscitation, 20 μg/kg over 10 min followed by 0.4 μg/kg/min for 220 min); and (3) equal volumes of saline as placebo.

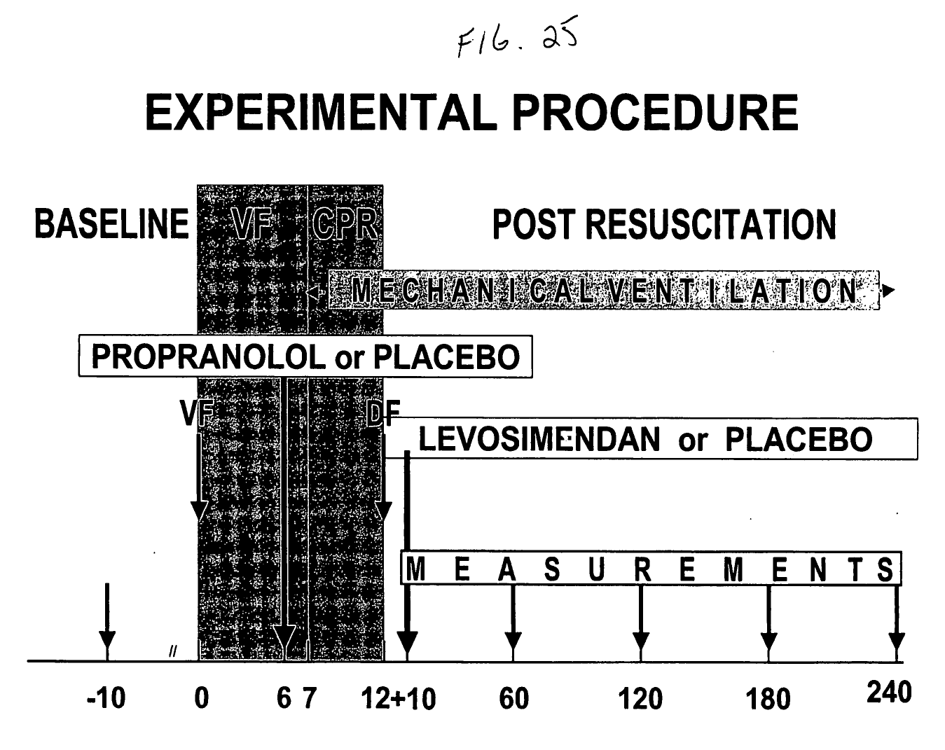

Measurements were obtained over an interval of 4 hours following resuscitation. The experimental procedures are summarized in FIG. 25. After 4 hours, animals were euthanized by intravenous injection of 150 mg kg⁻¹ pentobarbital. Autopsy was performed to document injuries to the bony thorax and the thoracic and abdominal viscera.

Measurements: Dynamic data, including aortic, right atrial (RAP), and pulmonary artery pressures (PAP), and end-tidal PCO₂ (P_{ET}CO₂), together with the electrocardiogram were continuously measured and recorded on a PC-based data acquisition system, supported by CODAS/WINDAQ hardware/software as previously described (14). A total of 16 channels were available for continuous recording at appropriate sampling frequencies for the studies proposed. The CPP was digitally computed, hemodynamic measurements and the electrocardiogram were displayed in real time.

Echocardiographic measurements were obtained with the aid a Hewlett-Packard Sonos 2500 echocardiographic system, utilizing a 5.5/7.5 Hz biplane Doppler transesophageal echocardiographic transducer with 4-way flexure (Model 21363A, Hewlett-Packard Co., Medical Products Group, Andover, Mass.). For the long axis, a 2- or 4-chamber view was obtained. Left ventricular end-systolic and -diastolic volumes were calculated by the method of discs (Acoustic Quantification Technology, Hewlett-Packard, Andover, Mass.). From these, ejection fractions (EF) and fractional area change (FAC) were computed. These measurements served as a quantitator of myocardial contractile function.

Aortic, mixed venous and great cardiac venous blood gases, hemoglobin and oxyhemoglobin were measured on 200 μL aliquots of blood with a stat profile analyzer (ULTRA C, Nova Biomedical Corporation, Waltham, Mass.) adapted for porcine blood. Arterial and great cardiac venous blood lactate was measured with a lactic acid analyzer (Model 23L, Yellow Springs Instruments, Yellow Springs, Ohio). These measurements were obtained at 10 min prior to cardiac arrest, at 10 min after ROSC and at hourly intervals thereafter, for a total of 4 hours. ST-T segment elevation was measured at 5 min after resuscitation and the total number of premature ventricular beats (PVB) over the 5 min interval that followed ROSC were counted. The total number and cumulative energies of shocks delivered were analyzed.

Statistical Analyses: All data are presented as means±standard deviation (SD). Differences of hemodynamic and metabolic measurements among groups were analyzed by ANOVA, including the Scheffe method for multiple comparison. A value of p<0.05 was regarded as significant.

Results: Baseline hemodynamic, blood gas and lactate measurements did not differ significantly among the three groups. Spontaneous circulation was restored in each animal. There were no significant differences in P_{ET}CO₂, blood gas analyses, and arterial blood lactate during and after CPR.

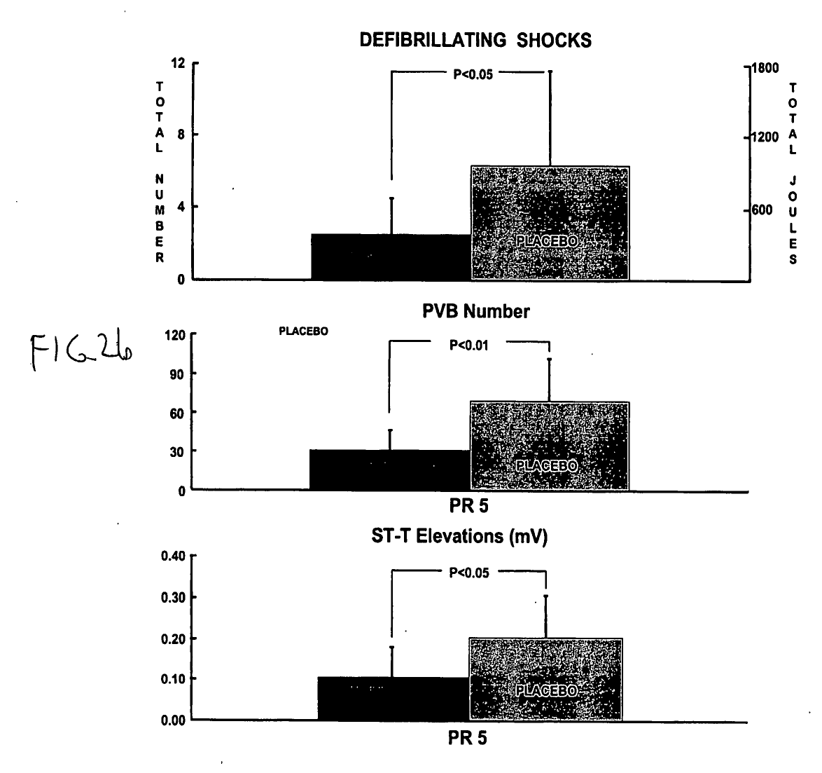

In confirmation of earlier observations, propranolol administered during CPR facilitated resuscitation with a significantly smaller number and significantly lesser total energies of electrical shocks. A significantly lesser number of postresuscitation premature ventricular beats and lesser postresuscitation ST segment elevation in ECG limb lead 2 were documented (FIG. 26).

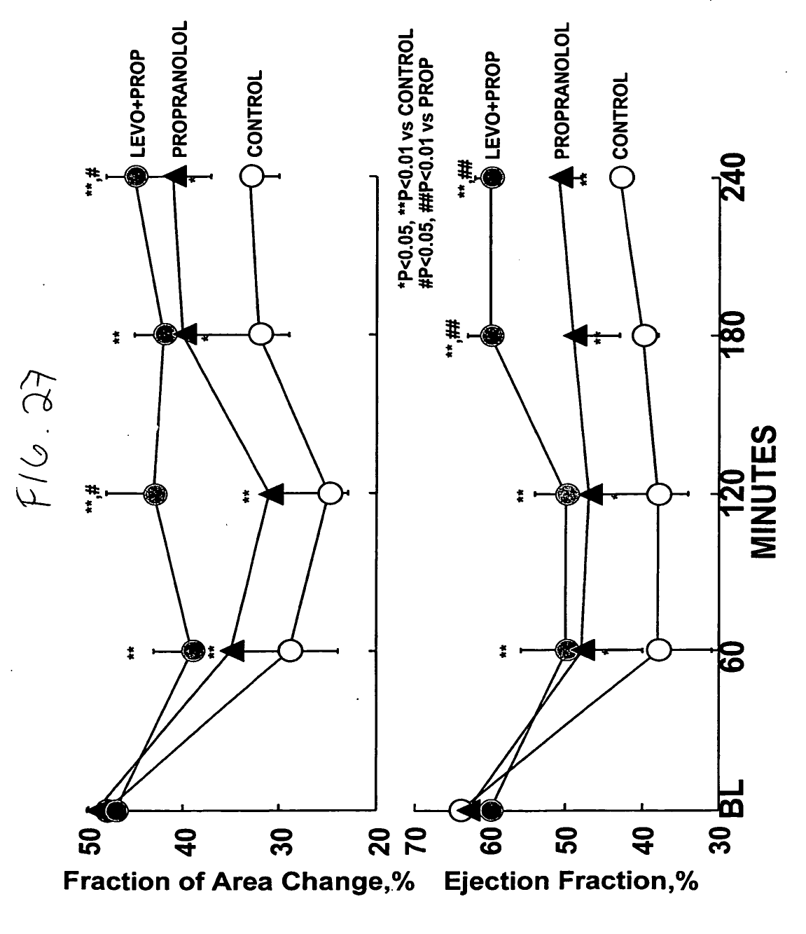

Postresuscitation ejection fractions and FAC were significantly increased after propranolol compared to saline placebo. When levosimendan was added during the early postresuscitation interval, additional and significant increases in EF and FAC were documented in comparison with propranolol alone as shown in FIG. 27.

The results of our experimental study extend an earlier report, which demonstrated that propranolol facilitated resuscitation and specifically electrical defibrillation, reduced the frequency of postresuscitation ectopy and moderated the severity of postresuscitation ischemic injury. When levosimendan was administered in the early postresuscitation interval, there was additional and significant improvement in myocardial contractile function.

What is claimed is:

1. In a method for restoring spontaneous circulation in a mammal in cardiac arrest wherein said method comprises the steps of administering cardiopulmonary resuscitation (CPR) and defibrillation shocks to said mammal, the improvement comprising administering to said mammal a therapeutically effective amount of a levosimendan compound for a pharmaceutically acceptable salt thereof.

2. The method of claim 1 wherein said step of administering occurs at the onset of administering said CPR.

3. The method of claim 1 wherein said levosimendan compound is administered in an amount of from about 0.06 to about 36 μg/kg/minute.

4. A method for reducing the frequency of defibrillation shocks applied to a mammal in cardiac arrest, the method comprising the steps of:
administering a therapeutically effective amount of a levosimendan compound or a pharmaceutically acceptable salt thereof to said mammal prior to applying said defibrillation shocks; and
applying said defibrillation shocks at a frequency sufficient to restore effective cardiac rhythm, wherein said frequency is reduced relative to the frequency established by a recognized standard of care protocol.

5. A method for reducing the frequency of defibrillation shocks applied to a mammal in cardiac arrest, the method comprising the steps of:
administering a therapeutically effective amount of a levosimendan compound or a pharmaceutically acceptable salt thereof to said mammal prior to applying said defibrillation shocks; and
applying said defibrillation shocks at a frequency sufficient to restore effective cardiac rhythm, wherein said frequency is reduced relative to the frequency of defibrillation shocks applied to a similar mammal in cardiac arrest which has not been treated with said levosimendan compound.

6. A method of reducing the energy of a defibrillation shock applied to a mammal in cardiac arrest, the method comprising the steps of:
administering a therapeutically effective amount of a levosimendan compound or a pharmaceutically acceptable salt thereof to said mammal prior to applying said defibrillation shock; and
applying said defibrillation shock to said mammal at said energy sufficient to restore effective cardiac rhythm, wherein said energy is reduced relative to said energy established by a recognized standard of care protocol.

7. A method of reducing the energy of a defibrillation shock applied to a mammal in cardiac arrest, the method comprising the steps of:
administering a therapeutically effective amount of a levosimendan compound or a pharmaceutically acceptable salt thereof to said mammal prior to applying said defibrillation shock; and
applying said defibrillation shock to said mammal at said energy sufficient to restore effective cardiac rhythm, wherein said energy is reduced relative to the energy applied to a similar mammal in cardiac arrest which has not been treated with said levosimendan compound.

8. The method of claim 7, further comprising the step of administering a therapeutically effective amount of an adrenergic receptor-blocking agent to said mammal prior to applying defibrillation energy to said mammal.

9. A method of treating myocardial dysfunction in a mammal in need thereof during or after resuscitation from cardiac arrest, comprising the step of administering to said mammal a therapeutically effective amount of a levosimendan compound or a pharmaceutically acceptable salt thereof.

10. The method of claims 1 or 4 or 5 or 6 or 7 or 9 wherein said levosimendan compound is levosimendan.

11. The method of claims 1 or 4 or 5 or 6 or 7 or 9 wherein said levosimendan compound is a metabolite of levosimendan.

12. The method of claims 1 or 4 or 5 or 6 or 7 or 9 wherein said step of administering comprises administering a continuous infusion of said levosimendan compound.

13. The method of claims 1 or 4 or 5 or 6 or 7 or 9 wherein said step of administering is parenteral.

14. The method of claims 1 or 4 or 5 or 6 or 7 or 9 wherein said parenteral administering is intravenous, endotracheal, intraarterial, transdermal or intracardiac.

15. The method of claims 1 or 4 or 5 or 6 or 7 or 9 wherein said levosimendan compound is administered in an amount of from about 0.01 to about 5.0 μg/kg/minute.

16. The method of claims 1 or 4 or 5 or 6 or 7 or 9 wherein said levosimendan compound is administered in an amount of from about 0.05 to about 0.4 μg/kg/minute.

17. The method of claims 1 or 4 or 5 or 6 or 7 or 9 wherein said levosimendan compound is administered in an amount of from about 0.1 μg/kg/minute.

18. The method of claims 1 or 4 or 5 or 6 or 7 or 9 wherein said mammal is a human.

19. The method according to claims 1 or 5 or 6 or 7 or 9 further comprising the step of administering a therapeutically effective amount of an adrenergic receptor-blocking agent to said mammal.

20. The method according to claim 4 further comprising the step of administering a therapeutically effective amount of an adrenergic receptor-blocking agent to said mammal.

21. The method according to claim 19, wherein said step of administering said adrenergic receptor-blocking agent occurs prior to said step of administering said levosimendan compound.

22. The method according to claim 19 wherein said adrenergic receptor-blocking agent is a beta adrenergic receptor-blocking agent or an alpha adrenergic receptor-blocking agent.

23. The method according to claim 22 wherein said beta adrenergic receptor-blocking agent is a beta-1 adrenergic receptor-blocking agent or a beta-2 adrenergic receptor-blocking agent.

24. The method according to claim 22 wherein said beta adrenergic receptor-blocking agent is propanolol, metoprolol, esmolol or atenolol.

25. The method according to claim 22 wherein said alpha adrenergic receptor-blocking agent is an alpha-1 adrenergic receptor-blocking agent.

26. The method according to claim 22 wherein the beta adrenergic receptor-blocking agent is carvedilol.

27. In a method for treating cardiac arrhythmia in a mammal in need thereof, wherein said method comprises the step of applying one or more defibrillation shocks to said mammal, the improvement comprising administering to said mammal, a therapeutically effective amount of a levosimendan compound or pharmaceutically acceptable salt thereof.

28. The method of claim 27 wherein said step of administering occurs after said applying one or more defibrillation shocks.

29. In a method for protecting organ function in a mammal subsequent to cardiac arrest, wherein said method comprises the step of restoring spontaneous circulation in said mammal, the improvement comprising administering to said mammal a therapeutically effective amount of a levosimendan compound or a pharmaceutically acceptable salt thereof.

30. The method of claim 29 wherein said organ function is brain organ function.

31. The method of claim 29 wherein said organ function is renal organ function.

32. The method of claim 29 wherein said organ function is hepatic organ function.
